SEQUENCE LISTING (4 sequences)

SEQ ID NO: 1 - protein, 13 aa, Artificial Sequence
  synthetic peptides
VCYDKSFPISHVR

SEQ ID NO: 2 - protein, 11 aa, Artificial Sequence
  synthetic peptides
SRPTEKTIFII

SEQ ID NO: 3 - protein, 18 aa, Artificial Sequence
  synthetic peptides
KRDPCHQVDCFLSRPTEK

SEQ ID NO: 4 - protein, 6 aa, Artificial Sequence
  synthetic peptides
  MOD_RES (4)..(4): 4Hyp
GAGXPY

(19) United States
(12) Patent Application Publication
(10) Pub. No.: US 2019/0054184 A1
(43) Pub. Date: Feb. 21, 2019

(54) Targeting Macrophages to Modulate Electrical Conduction in the Heart

(71) Applicant: The General Hospital Corporation, Boston, MA (US)

(72) Inventors: Maarten Hulsmans, Somerville, MA (US); Matthias Nahrendorf, Boston, MA (US); Ralph Weissleder, Peabody, MA (US)

(21) Appl. No.: 16/075,931

(22) PCT Filed: Feb. 13, 2017
(86) PCT No.: PCT/US17/17660

§ 371 Date: Aug. 6, 2018

Related U.S. Application Data
(60) Provisional application No. 62/294,765, filed on Feb. 12, 2016.

(51) Int. Cl. A61K 47/69; A61P 9/06; A61K 31/663

(52) U.S. Cl. CPC A61K 47/6911; A61K 31/663; A61P 9/06

(57) ABSTRACT

Compositions comprising a macrophage-targeted carrier and one or more therapeutic agents that modulate cardiac conductance, and methods of using the same for treating subjects with cardiac rhythm disorders, e.g., bradycardia or tachycardia.

### CLAIM OF PRIORITY

This application claims the benefit of U.S. Provisional Application Ser. No. 62/294,765, filed on Feb. 12, 2016. The entire contents of the foregoing are incorporated herein by reference.

### FEDERALLY SPONSORED RESEARCH

This invention was made with Government support under grant numbers NS084863, HL128264, HL114477, HL117829, HL092577, HL105780 and HL096576 awarded by the National Institutes of Health. The Government has certain rights in the invention.

### TECHNICAL FIELD

Described herein are compositions comprising a macrophage-targeted carrier and one or more therapeutic agents that modulate cardiac conductance, and methods of using the same for treating subjects with cardiac rhythm disorders, e.g., bradycardia or tachycardia.

### BACKGROUND

The cardiac conduction system coordinates the heart's contractile function. Electrical impulse propagation begins in the sinoatrial node and is followed by sequential activation of the atrium, atrioventricular (AV) node and ventricle. By providing the only electrical connection between the atria and ventricles, the AV node plays an essential role. As characterized by Sunao Tawara in 1906 (7), the AV node is located within the triangle of Koch at the base of the right atrium and contains a specialized subset of cardiomyocytes with a distinct action-potential morphology (8, 9). AV node conduction is slower than atrial or ventricular myocardium, giving rise to a delay that allows for ventricular filling during atrial contraction. Clinically, AV block delays or abolishes atrial impulse conduction to the ventricles, which can lead to hemodynamic deterioration, syncope and death if not treated with pacemaker implantation (10).

### SUMMARY

Recent work has recognized macrophages as an intrinsic part of the healthy working myocardium. They appear as spindle-like cells interspersed among myocytes, fibroblasts and endothelial cells (11-13). Cardiac healing after injury requires macrophages (14); however, the organ-specific functions of cardiac macrophages during physiological conditions are unknown. Here we report resident macrophages' abundance in the AV node and describe macrophages' essential contribution to AV conduction.

Thus, provided herein are compositions comprising a macrophage-targeted carrier and one or more therapeutic agents that modulate cardiac conductance, and optionally a pharmaceutically acceptable carrier. In some embodiments, the macrophage-targeted carrier is selected from the group consisting of microspheres/microparticles, liposomes, lipid nanoparticles, carbohydrate nanoparticles, dendrimers, exosomes, extracellular vesicles, carbon nanotubes, and polymersomes.

In some embodiments, the therapeutic agent decreases conductance. For example, in some embodiments, the therapeutic agent decreases gap junction communication, e.g., is endothelin-1, angiotensin II, Rotigaptide (ZP-123), peptide VCYDKSFPISHVR (SEQ ID NO: 1) corresponding to AA63-75 of E1 of Cx43; peptide SRPTEKTIFII (SEQ ID NO:2) corresponding to AA204-214 of E2 of Cx43; peptide KRDPCHQVDCFLSRPTEK (SEQ ID NO:3) corresponding to AA191-209 of E2 of Cx43), peptide AAP10 (H-Gly-Ala-Gly-Hyp-Pro-Tyr-CONH2), SEQ ID NO:4, cAAP10RG, AAPnat, or gap-134.

In some embodiments, the therapeutic agent is an anti-arrhythmic drug, e.g., a Ca²⁺ channel blocker; Na⁺ channel blocker; beta-adrenoceptor antagonists (beta-blockers); potassium-channel blocker; digoxin; or digitalis.

In some embodiments, the therapeutic agent increases conductance, e.g., is epinephrine, norepinephrine, dopamine, denopamine, dobutamine, salbutamol, atropine, isoproterenol, NS11021, naltriben, midefradil and NNC 50-0396, ICA-105574, PD-118057, NS1643, Pinacidil, 2-anilino-5-(2,4-dinitroanilino)benzenesulfonate; potassium channel agonists, e.g., NS-1619,1-EBIO, minoxidil, cromakalim, or levcromakalim, or a cation, e.g., K⁺, Na⁺, Ca²⁺, or Mg².

Also provided herein are methods for delivering a cardiac therapeutic agent to a subject, e.g., to the heart of a subject, e.g., for treating a subject having a cardiac rhythm disorder, the method comprising administering to the subject a therapeutically effective amount of a composition described herein.

For example, described are methods for treating a subject having tachycardia, comprising administering compositions comprising a macrophage-targeted carrier and one or more therapeutic agents that decreases conductance. For example, in some embodiments, the therapeutic agent decreases gap junction communication, e.g., is endothelin-1, angiotensin II, Rotigaptide (ZP-123), peptide VCYDKSFPISHVR (SEQ ID NO: 1) corresponding to AA63-75 of E1 of Cx43; peptide SRPTEKTIFII (SEQ ID NO:2) corresponding to AA204-214 of E2 of Cx43; peptide KRDPCHQVDCFLSRPTEK (SEQ ID NO: 3) corresponding to AA191-209 of E2 of Cx43), peptide AAP10 (H-Gly-Ala-Gly-Hyp-Pro-Tyr-CONH2), SEQ ID NO:4, cAAP10RG, AAPnat, or gap-134. In some embodiments, the therapeutic agent is an anti-arrhythmic drug, e.g., a Ca²⁺ channel blocker; Na⁺ channel blocker; beta-adrenoceptor antagonists (beta-blockers); potassium-channel blocker; digoxin; or digitalis.

Also described are methods for treating a subject having bradycardia or a conductance block, comprising administering a macrophage-targeted carrier and one or more therapeutic agents that decreases conductance. For example, in some embodiments, the therapeutic agent increases conductance, e.g., is epinephrine, norepinephrine, dopamine, denopamine, dobutamine, salbutamol, atropine, isoproterenol, or a conductance-increasing amount of a cation, e.g., K⁺, Na⁺, Ca²⁺, or Mg²⁺.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIGS. 1A-1D. Resident Cardiac Macrophages in the AV Node.

FIG. 1A. Volumetric reconstruction of confocal microscopy after optical clearing of the atrioventricular (AV) node in a Cx₃cr1^{GFP/+} mouse stained with HCN4. The node is orientated along the AV groove extending from the compact node (CN) into the proximal His bundle. Dashed square indicates the lower nodal or AV bundle. CFB, central fibrous body; IAS and IVS, interatrial and interventricular septum.

FIG. 1B. Higher magnification of dashed square in FIG. 11A.

FIG. 1C. 3D rendering of GFP⁺ macrophages in the AV bundle.

FIG. 1D. Electron microscopy of a DAB⁺ macrophage in AV node of Cx₃cr1^{GFP/+} mouse stained with a primary antibody for GFP. Arrow indicates nucleus, arrowheads indicate cellular processes.

FIGS. 2A-2D. The AV Node Enriches for Macrophages.

FIG. 2A. Flow cytometric macrophage quantification in microdissected AV node and left ventricular (LV) free wall of C57BL/6 mice. (Left) Representative flow cytometry plots; (right) number of macrophages per mg of heart tissue. White bars represent LV free wall macrophages and grey bars represent AV node macrophages. Data are mean±SEM, n=12 mice of 4 independent experiments, **p<0.01, Student's t test.

FIG. 2B. Expression of CD64, CX₃CR1, CD11c and CD103 on AV node and LV free wall macrophages. Representative histograms of 4 mice are shown. Gray, isotype control antibody.

FIG. 2C. Macrophage chimerism in the LV free wall (white bars) and AV node (grey bars) and monocyte chimerism in the blood (black bars) of C57BL/6 mice that had been joined in parabiosis with Cx₃cr1^{GFP/+} mice for 12 weeks (mean±SEM, n=3 [AV node] and n=7 [LV free wall and blood] of 2 independent experiments).

FIG. 2D. (Top) Workflow; (bottom) Heat map of expression levels (cpm, counts per million) among top 200 overdispersed genes from RNA-seq data of 76 AV node macrophages. Unsupervised clustering reflects three macrophage subsets according to expression levels of H2 and Ccr2 (MHCII^{low} CCR2^{low}; MHCII^{high}CC2^{high}; MHCII^{high}CCR2^{low}).

FIGS. 3A-3B. Macrophages in the Human AV Node.

FIG. 3A. Masson's Trichrome stain of human tissue to identify the AV node. IAS and IVS, interatrial and interventricular septum.

FIG. 3B. Immunohistochemical stain for CD68 in human working myocardium and AV node. Data are mean±SEM, n=20 to 30 high-power fields per section, ****p<0.0001, Student's t test.

FIGS. 4A-4L. AV Node Macrophages Couple to Conducting Cardiomyocytes and Alter Their Electrophysiological Properties

FIG. 4A. Relative connexin (Cx) expression levels in FACS-purified AV node macrophages by qPCR (n=4 to 6 of 2 independent experiments).

FIG. 4B. Cx43 levels by qPCR in macrophages FACS-sorted from AV node and peritoneum. n=6 to 9 of 2 independent experiments.

FIG. 4C. Whole-mount immunofluorescence microscopy of AV lower nodal area from a Cx₃cr1^{GFP/+} mouse stained with Cx43 and HCN4. Arrowheads indicate Cx43 colocalization with GFP⁺ macrophages.

FIG. 4D. Electron microscopy image of a direct membrane contact (arrow) of a DAB⁺ macrophage and a cardiomyocyte in AV node tissue of a Cx₃cr1^{GFP/+} mouse stained for GFP. The nodal cardiomyocyte is characterized by its typical high mitochondrial content and junctional contact with the neighboring myocyte (arrowhead).

FIG. 4E. Immunofluorescence image of a co-cultured desmin neonatal mouse cardiomyocyte and GFP⁺ cardiac macrophage stained with Cx43 (arrow), illustrating setup for patch clamp experiments. The cells are grown on cover slips coated with fibronectin in a line pattern.

FIG. 4F. Immunofluorescence images of dextran diffusion during whole-cell patch clamp with a dextran-loaded pipette. (Top) Arrowhead indicates GFP cardiac macrophage; (bottom) Texas Red dextran diffusion into macrophage.

FIG. 4G. Spontaneous recordings of solitary cardiac macrophages (n=20) and macrophages attached to cardiomyocytes (n=43) by whole-cell patch clamp.

FIG. 4H. Resting membrane potential of solitary cardiac macrophages (n=20) and macrophages attached to cardiomyocytes (n=43) by whole-cell patch clamp. Data are mean±SEM from 13 independent experiments, **p<0.01, nonparametric Mann-Whitney test. Rhythmic depolarization was observed in 10/43 macrophages attached to cardiomyocytes.

FIG. 4I. Resting membrane potential of solitary cardiomyocytes (n=13) and cardiomyocytes coupled to macrophages before (n=14) and after (n=7) addition of the Cx43 inhibitor Gap26 (SEQ ID NO: 1). Data are mean±SEM from 3 independent experiments, *p<0.05 and **p<0.01, Kruskal-Wallis test followed by Dunn's posttest.

FIG. 4J. Mathematical modeling of AV bundle cardiomyocyte membrane potential uncoupled or coupled to one, two or four cardiac macrophages at a junctional conductance of 1 nS.

FIG. 4K Computational modeling of resting membrane potential of an AV bundle cardiomyocyte coupled to an increasing number of cardiac macrophages.

FIG. 4L. Computational modeling of action potential duration of an AV bundle cardiomyocyte coupled to an increasing number of cardiac macrophages.

FIGS. 5A-5D. Optogenetics Stimulation of AV Node Macrophages Improves Nodal Conduction.

FIG. 5A. Experimental outline. Hearts of Cx₃cr1^{wt/CreER} (control) or tamoxifen-treated Cx₃cr1^{wt/CreER} ChR2^{wt/fl} (Cx₃cr1 ChR2) mice were perfused in a Langendorff setup. Recording and pacing electrodes were connected to the heart and illumination with a fiber optic cannula was focused on the AV node.

FIG. 5B. Images illustrating the optogenetics experimental setup during a light off and on cycle.

FIG. 5C. Representative ECG recordings from a Cx₃cr1 ChR2 heart illustrating the number of conducted atrial stimuli between two non-conducted impulses of a Wenckebach period during light off and on cycles. Arrows indicate failure of conduction leading to missing QRS complexes. Stim, stimulation.

FIG. 5D. Representative bar graph of a Cx₃cr1 ChR2 heart showing the number of conducted atrial stimuli between two non-conducted impulses of a Wenckebach period during light off and on cycles. Data are mean±SEM, **p<0.01, nonparametric Mann-Whitney test.

FIGS. 6A-6H. Cx43 Deletion in Macrophages and Congenital Lack of Macrophages Delay AV Conduction.

FIG. 6A. Experimental outline of the electrophysiological (EP) study performed on mice lacking Cx43 in macrophages.

FIG. 6B. AV node effective refractory period at 120 ms pacing frequency, and pacing cycle lengths at which Wenckebach conduction, 2:1 conduction and ventriculo-atrial (VA) Wenckebach conduction occurred in control (n=5 to 9) and Cx₃cr1 Cx43^{-/-} (n=6 to 8) mice. Data are mean±SEM, 2 independent experiments, *p<0.05 and **p<0.01, Student's t test and nonparametric Mann-Whitney test.

FIG. 6C. Surface ECG from control and Cx₃cr1 Cx43^{-/-} mice illustrating the Wenckebach cycle length. Arrows indicate missing QRS complexes. Stim, stimulation.

FIG. 6D. Flow cytometric quantification of AV node macrophages in control and Cx₃cr1 Cx43^{-/-} mice. Data are mean±SEM, n=6 mice per group, nonparametric Mann-Whitney test.

FIG. 6E. Immunofluorescence images of control and Cx₃cr1 Cx43^{-/-} AV node stained for CD68 and HCN4.

FIG. 6F. Quantification of AV node macrophages in control (n=5) and Csf1^{op} (n=4) mice by flow cytometry. Data are mean±SEM, 3 independent experiments, *p<0.05, nonparametric Mann-Whitney test.

FIG. 6G. Immunofluorescence image of a Csf1^{op} AV node stained for CD68 and HCN4.

FIG. 6H. AV node effective refractory period at 120 ms pacing frequency, and pacing cycle lengths at which Wenckebach and 2:1 conduction occurred in control (n=6) and Csf1^{op} (n=5) mice. Data are mean±SEM, 3 independent experiments, **p<0.01, nonparametric Mann-Whitney test.

FIGS. 7A-7D. Macrophage Ablation Induces AV Block.

FIG. 7A. Experimental outline. DT, diphtheria toxin.

FIG. 7B. Flow cytometric quantification of AV node macrophages three days after intraperitoneal injection of DT into C57BL/6 and Cd11b^{DTR} mice. Data are mean±SEM, n=6 mice per group, **p<0.01, nonparametric Mann-Whitney test.

FIG. 7C. Onset of first degree AV block in Cd11b^{DTR} (n=6) and C57BL/6 (n=10) animals after DT injection (2 independent experiments, ****p<0.0001, Mantel-Cox test).

FIG. 7D. Telemetric ECG recordings before and after DT injection in Cd11b^{DTR} mice. Arrows indicate non-conducted P waves in second degree AV block.

FIG. 8. Histological Macrophage Quantification in AV Bundle and LV Free Wall. Percentage of positive staining per region of interest (ROI). Data are mean±SEM, n=3-6 mice of 2 independent experiments, **p<0.01, Kruskal-Wallis test followed by Dunn's posttest.

FIGS. 9A-9F. Identification of Three Subsets of AV Node Macrophages.

FIG. 9A. Grouping of AV node macrophages according to their expression levels of H2 and Ccr2.

FIG. 9B. Principal component (PC) analysis of 76 single-cell samples based on expression levels of overdispersed genes, color-coded according to the three subsets in FIG. 9A.

FIG. 9C. Variables factor map of the top 200 overdispersed genes highlighting H2 and Ccr2. The arrow tip denotes the correlation coefficients of the respective gene with the first two principal components.

FIG. 9D. Venn diagram illustrating the shared expression profile of conduction-related genes for the three AV node macrophage subsets by single-cell RNA-seq.

FIG. 9E. Ion channel expression by qPCR in FACS-purified macrophages and whole AV node (n=4 to 9 of 2 independent experiments). P, peritoneum; mac, macrophage.

FIG. 9F. Gene set enrichment analysis shows that expression of genes involved in cardiac conduction (GO:0061337) is higher in cardiac macrophages than in brain- and spleen-derived macrophages (q value<0.05).

FIGS. 10A-10D. Purity of FACS-sorted Macrophages and Cx43 Contact Points between AV Node Macrophages and Cardiomyocytes.

FIG. 10A. Macrophage gene expression by qPCR in FACS-purified macrophages and whole AV node (n=5 to 9 of 2 independent experiments). P, peritoneum; mac, macrophage.

FIG. 10B. Cardiomyocyte-specific gene expression by qPCR in FACS-purified macrophages and whole AV node (n=5 to 9 of 2 independent experiments). P, peritoneum; mac, macrophage.

FIG. 10C. Number of Cx43⁺ contact points between adjacent HCN4⁺ cardiomyocytes and between CX₃CR1⁺ macrophages and HCN4⁺ cardiomyocytes in the AV bundle. Data are mean±SEM, n=27-31 in 5 mice.

FIG. 10D. Whole-mount immunofluorescence microscopy of the human AV bundle stained with CD163 and Cx43. Arrowheads indicate Cx43 colocalization with macrophages. Autofluorescence signal (AF) was used for visualization of cell morphology.

FIGS. 11A-11F. Electrophysiological Properties of Cardiac Macrophages and Cardiomyocytes.

FIG. 11A. Representative spontaneous recordings of cardiac macrophages attached to co-cultured neonatal mouse cardiomyocytes show no activity (n=23), irregular depolarization (n=10) and regular depolarization (n=10).

FIG. 11B. Resting membrane potentials of cardiac macrophages attached to co-cultured neonatal mouse cardiomyocytes show no activity (n=23), irregular depolarization (n=10) and regular depolarization (n=10). Data are mean±SEM from 13 independent experiments, *p<0.05, Kruskal-Wallis followed by Dunn's posttest.

FIG. 11C. Immunofluorescence images of a GFP⁺ cardiac macrophage and cardiomyocyte both loaded with ANNINE-6plus voltage-sensitive dye. Arrow and arrowhead demark the positions of simultaneous line-scan data acquisition in macrophage and cardiomyocyte, respectively.

FIG. 11D. Spontaneous, simultaneous recordings of action potential-related fluorescence changes (ΔF/F₀) in the cardiac macrophage and cardiomyocyte depicted in FIG. 11C.

FIG. 11E. Resting membrane potential of solitary cardiomyocytes (n=3) before and after adding the Cx43 inhibitor Gap26. Data are mean±SEM from 3 independent experiments, Wilcoxon rank-sum test.

FIG. 11F. Simulated membrane potential of an AV bundle cardiomyocyte uncoupled or coupled to one cardiac macrophage at increasing junctional conductance (G_{gap}).

FIGS. 12A-12C. CreER and ChR2 are Specifically Expressed in CX₃CR1⁺ Cardiac Macrophages.

FIG. 12A. YFP target-to-background ratio (TBR) of cardiomyocytes and CX₃CR1⁺ macrophages (targets) in comparison with ECs (background). Data are mean±SEM, n=5 to 20 z-stack images per group, **p<0.01, nonparametric Mann-Whitney test.

FIG. 12B. Diagram illustrating the mathematical model of macrophage-mediated passive action potential conduction. Two strands of 10 cardiomyocytes with intercellular conductance of 167 nS are connected via one macrophage. The outer half of the proximal (left) cardiomyocyte strand is stimulated with 2 nA per cell at 3 Hz and the minimum heterocellular junctional conductance (G_{gap}) that can support macrophage-mediated passive conduction of sufficient amplitude to stimulate an action potential at the distal strand is determined by modeling.

FIG. 12C. Minimum junctional conductance between macrophage and cardiomyocyte strands sufficient to bridge action potential propagation between cardiomyocyte strands that are connected via a single macrophage only. The required junctional conductance decreases with macrophage depolarization, i.e. the likelihood of conduction increases with a more positive macrophage resting membrane potential.

FIGS. 13A-13C. Cx43 is Specifically Depleted in CX₃CR1⁺ Cardiac Macrophages.

FIG. 13A. PCR analysis of FACS-purified Cx₃cr1^{wt/wt} and Cx₃cr1^{wt/CreER} cardiac macrophages seven days post-tamoxifen for the presence of wild-type (Cx43^{wt}) and conditional undeleted (Cx43^{ft}) or deleted (Cx43^{Δ}) Cx43 alleles.

FIG. 13B. Cx43 mRNA levels in FACS-purified control and Cx₃cr1 Cx43^{-/-} cardiac macrophages by qPCR (mean±SEM, n=3 mice per group).

FIG. 13C. Western blot showing Cx43 expression in heart tissue of control and Cx₃cr1 Cx43^{-/-} mice. Data are mean±SEM, n=4 mice per group, nonparametric Mann-Whitney test.

FIGS. 14A-14F. AV Block in Cd11b^{DTR} mice is Not a Consequence of Local Cell Death, Electrolyte Imbalance, Diphtheria Toxin Toxicity or Autonomic Nervous Imbalance.

FIG. 14A. Immunofluorescence image of the AV node in macrophage-depleted Cd11b^{DTR} mice stained with HCN4 and TUNEL.

FIG. 14B. Blood electrolytes of Cd11b^{DTR} and C57BL/6 animals three days after DT injection (mean±SEM, n=5 mice per group, nonparametric Mann-Whitney test).

FIG. 14C. Surface ECG of Cd11b^{DTR} and Cx₃cr1^{GFP/+} parabionts three days after DT injection (mean±SEM, n=3 parabiosis pairs).

FIG. 14D. Surface ECG of Cd11b^{DTR} mice with second and third degree AV block after intravenous isoproterenol, atropine or epinephrine administration. Arrows indicate non-conducted P waves in second degree AV block.

FIG. 14E. Heat map of expression values (cpm) among the top 50 dysregulated genes in microdissected AV nodes of control (n=3), Cx₃cr1 Cx43^{-/-} (n=3) and macrophage-depleted Cd11b^{DTR} (n=3) mice by RNA-seq.

FIG. 14F. Gene set enrichment analysis shows that expression of genes involved in cardiac conduction (GO:0061337) is lower in macrophage-depleted AV nodes than in control AV nodes (q value<0.0001).

### DETAILED DESCRIPTION

Studies in the late 19^{th} century first described macrophages as phagocytic cells that consume foreign bodies and pathogens (1). These hematopoietic cells of myeloid lineage populate all tissues and have important roles in immune defense. Resident macrophages may also control tissue homeostasis in an organ-specific manner (2). For instance, macrophages contribute to thermogenesis regulation in adipose tissue (3), iron recycling in the spleen (4) and synaptic pruning in the brain (5). These data highlight macrophages' functional diversity and emphasize their capability to execute tissue-specific functions beyond traditional roles in host defense (6).

The presence of numerous macrophages resident in the myocardium has only recently gained recognition (11-13). This discovery triggered the quest to decipher macrophages' roles in organ homeostasis beyond their long-recognized functions in innate immunity and host defenses (14). Here we report on intra-organ macrophage heterogeneity in the heart; more specifically we show that macrophages are electrically coupled to specialized conducting cells in the AV node and that macrophage loss results in fatal AV block.

Studies in the 1970s and 1980s already observed macrophage depolarization, in vitro, in mouse peritoneal macrophages and in human alveolar and monocyte-derived macrophages (19, 20, 21). Work from this era concluded that macrophages exhibit complex electrophysiological properties often associated with excitable cells and that electrical signals may contribute to macrophage functions such as chemotaxis, receptor-ligand interactions and phagocytosis. Action potentials in paced macrophages are sodium-insensitive and calcium dependent (22). Here we demonstrate that cardiomyocytes can drive cardiac macrophages' rhythmic depolarization via Cx43-containing gap junctions. Gap junction-mediated intercellular communication also contributes to macrophage immune functions, including Cx43-dependent antigen peptide transfer from macrophages to antigen-presenting cells (23, 24).

The presence of Cx43-containing gap junctions in the AV node has previously been reported in humans and rabbits (25-27). Homozygous Cx43^{-/-} mice are not viable, but a 50% reduction of Cx43 in heterozygous Cx43^{+/-} mice associates with slower ventricular conduction and a retrograde Wenckebach conduction at slower pacing rates (28). This latter finding parallels the phenotype we observed in Cx₃cr1 Cx43^{-/-} mice.

Like the heart, the brain is an electrically active organ that contains macrophages. In the brain, astrocytes are linked by gap junctions and communicate with each other and neurons via release of neurotransmitters in a calcium-dependent manner, constituting a form of excitability (29). Microglia, the brain's resident macrophages, regulate astrocyte-mediated modulation of excitatory neurotransmission (30). These insights provide a basis for understanding pathological microglia activation and synaptic dysfunction in brain diseases. The influence of macrophages on information transfer in the brain bears some similarity to the discoveries described here.

Clinically, AV block is a common indication for pacemaker implantation, yet up to 60% of AV blocks occur for unknown reasons (31). Understanding macrophages' contributions to conduction abnormalities yields new pathophysiologic insight and suggests novel therapeutic strategies that could obviate the expense and complications associated with the three million pacemakers currently implanted worldwide.

Methods for Treating Cardiac Rhythm Disorders

The present disclosure provides for delivering cardiac therapeutic agents to the heart, e.g., for treating cardiac rhythm disorders, with macrophage targeted therapeutics. It was not previously known that a) macrophages reside in the electrical conduction system including the AV node, and b) that they functionally influence cardiac conduction (as shown herein, macrophage depletion causes AV block). Macrophage-targeted interventions may, depending on desired action, increase or decrease cardiac conduction.

The methods include modulating macrophage presence and phenotype with macrophage-targeted cardiac therapeutics (e.g., delivering therapeutic agents such as antibodies, growth factors, or small molecule drugs using particulate delivery vehicles including nanoparticles, microparticles, or liposomes) that will affect cardiac conduction. Generally, the methods include administering a therapeutically effective amount of macrophage-targeted therapeutics as described herein to a subject who is in need of, or who has been determined to be in need of, such treatment.

As used in this context, to “treat” means to ameliorate at least one symptom of the cardiac rhythm disorder. Administration of a therapeutically effective amount of a composition described herein for the treatment of a condition associated with bradycardia or a cardiac conduction block will result in increased conduction, while administration of a therapeutically effective amount of a composition described herein for the treatment of a condition associated with tachycardia or hyperconduction will result in decreased conduction/conduction block. Increasing conduction is important in patients with bradycardia or a cardiac conduction block, for instance AV block. Administration of atropine or isoproterenol infusion may improve AV conduction, e.g., where bradycardia is caused by a proximal AV block (located in the atrioventricular node) but may be contraindicated if the block is in the His-Purkinje system. Decreasing conduction is important in patients with tachycardia, for instance atrial fibrillation or flutter. Specific therapies for specific arrhythmias are known in the art; see, e.g., Zipes et al., Circulation. 2006 Sep. 5; 114(10):e385-484, which is incorporated by reference herein.

One of skill in the art can readily identify those subjects who would benefit from treatment with the methods described herein. For example, an EKG or ECG can be used to detect the presence of abnormal cardiac rhythms and thus increased or decreased conductance.

Therapeutic Agents for Modulating Conductance

The methods described herein can include modulating, e.g., increasing or decreasing conductance.

Gap junction communication between macrophages and cardiomyocytes can be modulated (e.g., decreased) using gap junction modulating drugs delivered by cargo vehicles as described above, e.g., endothelin-1, angiotensin II, Rotigaptide (ZP-123), peptide VCYDKSFPISHVR (SEQ ID NO:1) corresponding to AA63-75 of E1 of Cx43; peptide SRPTEKTIFII (SEQ ID NO:2) corresponding to AA204-214 of E2 of Cx43; peptide KRDPCHQVDCFLSRPTEK (SEQ ID NO:3) corresponding to AA191-209 of E2 of Cx43), peptide AAP10 (H-Gly-Ala-Gly-Hyp-Pro-Tyr-CONH2), SEQ ID NO:4, cAAP10RG, AAPnat, and gap-134, which can be used to decrease conduction for the treatment of cardiac arrhythmias e.g., atrial fibrillation. Rotigaptide is a peptide analog that has been shown to increase gap junction intercellular conductance in cardiac muscle cells (Shiroshita-Takeshita et al. (2007), Circulation. 115: 310-318). Gap-134 is a non-peptide analogue of AAP10. See, e.g., Dhein, Peptides 23:1701-1709 (2002).

Cardiomyocyte conduction can also be altered (reduced) using anti-arrhythmic drugs such as Ca²⁺ channel blockers (a number of which are known, including amlodipine (Norvasc), diltiazem (Cardizem LA, Tiazac), felodipine (Plendil), isradipine (Dynacirc), nifedipine (Adalat, Procardia), nicardipine (Cardene), nimodipine (Nimotop), nisoldipine (Sular), and verapamil (Covera-HS, Verelan PM, Calan)); Na⁺ channel blockers (e.g., quinidine, procainamide, disopryamide, lidocaine, tocainide, mexiletine, flecainide, propafenone, or moricizine); beta-adrenoceptor antagonists (beta-blockers), e.g., non-selective β1/β2 antagonists (e.g., carteolol, carvedilol, labetalol, nadolol, penbutolol, pindolol, propranolol, sotalol, or timolol) or β1-selective antagonists (e.g., acebutolol, atenolol, betaxolol, bisoprolol, esmolol, metoprolol, or nebivolol); potassium-channel blockers (e.g., amiodarone, dronedarone, bretylium, sotalol, ibutilide, or dofetilide); digoxin, and digitalis).

Alternatively, to increase conductance (e.g., to relieve AV block), drugs such as epinephrine, norepinephrine, dopamine, denopamine, dobutamine, salbutamol, atropine, isoproterenol, can be used, or cations can be delivered to influence macrophage membrane potential and therefore change cardiac conduction. These ions include K+, Na+, Ca2+, and Mg2+, which can be delivered using cargo vehicles and macrophage-based delivery vehicles (e.g., doped anion exchange polymers or nanoparticles with large payloads of these elemental cations); concentrations can be varied to increase or decrease conductance. The small molecule NS11021 (1-[3,5-bis(trifluoromethyl)phenyl]-3-[4-bromo-2-(2H-tetrazol-5-yl)phenyl]thiourea) is a potent and specific activator of Ca2+-activated big-conductance K+ channels. (Bentzen, B H. et al. (2007), Molecular Pharmacology. 72(4):1033-44). Similar conductance activators also include 2-amino benzimidazole relatives of the TRPM7 inhibitor NS8593 (US2010035951) that work as agonists or activators of the said channels. These can include naltriben, midefradil and NNC 50-0396 that act as positive regulators or activators of TRPM7. Midefradil and NNC 50-0396 are Mg2+-regulated (Schafer, S. et al. (2015), Pflugers Archives. 1-12). ICA-105574, a substituted benzamide, is a recently developed hERG activator that ameliorates cardiac conductance and prevents arrhythmias induced by cardiac delayed repolarization (Meng, J. et al. (2013), European Journal of Pharmacology. 718 (1-3), 87-97) and PD-118057 is a 2-(phenylamino) benzoic acid that enhances open probability of channels that determines cardiac conductivity enhancement and may prevent arrhythmia. NS1643 (Diness, T G. et al. (2008), Cardiovascular Research. 79(1): 61-69) and Hexachlorophene (Zheng, Y. et al. (2012), PLoS One. 7(12): e51820) are also known cardiac conductance activators. Pinacidil is a known potassium channel opener that also activates cardiac conductance (Cao, S. et al. (2015), Molecular Medicine Reports. 12(1): 829-836). Further non-limiting examples of conductance channel agonists include sodium; 2-anilino-5-(2,4-dinitroanilino)benzenesulfonate (US20140303226); potassium channel agonist (other than bradykinin or a bradykinin analog), such as NS-1619,1-EBIO, a guanylyl cyclase activator, a guanylyl cyclase activating protein, minoxidil, cromakalim, or levcromakalim (U.S. Pat. No. 7,018,979).

Additional cardiac therapeutics are known in the art; see, e.g., Zipes et al., Circulation. 2006 Sep. 5; 114(10):e385-484, which is incorporated by reference herein.

Macrophage-Targeting Carriers

As noted above, delivery vehicles including microspheres/microparticles, liposomes, lipid nanoparticles, carbohydrate nanoparticles, nanoparticles, dendrimers, exosomes, extracellular vesicles, carbon nanotubes, and polymersomes can for example be used as macrophage-avid cargo vehicles to carry therapeutic agents to and into macrophages in the heart.

The phagocytic nature of macrophages makes them readily targetable; for example, nano- or micro-particles comprising a metal core (e.g., iron oxide or gold, e.g., crosslinked dextran iron oxide nanoparticles) have been demonstrated to be taken into cardiac macrophages (see, e.g., Weissleder et al., Nature Materials 13:125-138 (2014)). A number of nanocarriers have been described for macrophage-targeted drug delivery; see, e.g., Jain et al., Expert Opin Drug Deliv. 2013 March; 10(3):353-67, especially Table 1 and the references cited therein.

Nano- or micro-particles coated with ligands to macrophage surface receptors such as dextran (see, e.g., Choi et al., the ACS journal of surfaces and colloids. 2010; 26:17520-7; Lim et al., Nanotechnology. 2008; 19: 375105), tuftsin (Jain and Amiji, Biomacromolecules. 2012; 13:1074-85); mannose (Kelly et al., Journal of drug delivery. 2011; 2011: 727241), and hyaluronate (Chellat et al., Biomaterials. 2005; 26: 7260-75) can be used to actively target macrophages. See, e.g., Patel and Janjic, Theranostics 2015; 5(2): 150-172. Engineered PLGA nanoparticles, surface-functionalized gelatin nanoparticles, and Hydrophilic albumin microspheres have been used to deliver Amphotericin B (Nahar et al., Pharm Res 2009; 26:2588-98; Nahad et al., J Drug Target 2010; 18(2):93-105; Sanchez-Brunete et al., Antimicrob Agents Chemother 2004; 48(9):3246-52). Mannose-conjugated solid lipid nanoparticles were used to deliver Rifabutin (Nimje et al., J Drug Target 2009; 17:777-87).

Dendrimers, e.g., poly(propyleneimine) (PPI) dendrimers including mannose-conjugated PPI dendrimers can also be used to target drugs to macrophages; see, e.g., Kumar et al., J Drug Target 2006; 14(8):546-56); Mishra et al., Pharmazie 2010; 65(12):891-5; Dutta et al., Eur J Pharm Sci 2008; 34:181-9; and Jain et al., Expert Opin Drug Deliv. 2013 March; 10(3):353-67.

Liposomes have also been described for use in delivering drugs to macrophages; see, e.g., Kelly et al., Journal of drug delivery. 2011; 2011:727241; targeting can be enhanced by inclusion of ligands as noted above. Ciprofloxacin has been delivered incorporated into mannosylated liposomes (Chono et al., J Control Release 2008; 127:50-8). Other liposomal coatings include Oligomannose; Polyethylene glycol (PEG) (to increase half-life); and Hyaluronan. See Jain et al., Expert Opin Drug Deliv. 2013 March; 10(3):353-67, Table 1. In some embodiments, small (<100 nm) negatively charged liposomes (e.g., comprising neutral 1,2-distearoylsn-glycero-3-phosphocholine (DSPC), anionic distearoylphophatidylglycerol (DSPG), and cholesterol at a molar ratio of about 3:1:2) can be used (see Kelly et al. 2011).

Niosomes, which are non-ionic surfactant-based uni- and multi-lamellar vesicles previously used to deliver therapeutics for cancer and tuberculosis (see, e.g., Jain et al., Expert Opin Drug Deliv. 2013 March; 10(3):353-67; Gaikwad et al., Cancer Biother Radiopharm 2000; 15(6):605-15; Gude et al., Cancer Biother Radiopharm 2002; 17:183-9; Singh et al., Trop J Pharm Res 2011; 10(2):203-10), can also be used to target macrophages, as can carbon nanotubes (see, e.g., Iijima, Nature 1991; 354:56-8; Jain et al., Nanotoxicology 2007; 1(3): 167-97; Mehra et al., Crit Rev Ther Drug Carr Syst 2008; 25(2):169-206; Jain et al., Nanomed Nanotech Biol Med 2009; 5(4):432-42 (Galactose conjugated multi walled carbon nanotubes); Prajapati et al., J Antimicrob Chemother 2011; 66:874-9). Polymersomes, which are polymeric vesicles made of amphiphilic block copolymers that self-assemble in aqueous solutions, have an aqueous center separated from outer fluids by the hydrophobic copolymer membranes (Jain et al., Expert Opin Drug Deliv. 2013 March; 10(3):353-67).

Pharmaceutical Compositions and Methods of Administration

The methods described herein include the use of pharmaceutical compositions comprising at least one active ingredient that modulates conductance, and a macrophage-targeting carrier, e.g., microspheres/microparticles, liposomes, lipid nanoparticles, carbohydrate nanoparticles, dendrimers, exosomes, extracellular vesicles, carbon nanotubes, and polymersomes, e.g., wherein the active ingredient is linked to or encapsulated within or otherwise conjugated to the carrier.

Pharmaceutical compositions typically include a pharmaceutically acceptable carrier. As used herein the language “pharmaceutically acceptable carrier” includes saline, solvents, dispersion media, coatings, antibacterial and antifungal agents, isotonic and absorption delaying agents, and the like, compatible with pharmaceutical administration.

Pharmaceutical compositions are typically formulated to be compatible with its intended route of administration. Examples of routes of administration include parenteral, e.g., intravenous, intradermal, subcutaneous, oral (e.g., inhalation or ingestion), transdermal (topical), transmucosal, and rectal administration.

Methods of formulating suitable pharmaceutical compositions are known in the art, see, e.g., Remington: The Science and Practice of Pharmacy, 21st ed., 2005; and the books in the series Drugs and the Pharmaceutical Sciences: a Series of Textbooks and Monographs (Dekker, NY). For example, solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use can include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.), phosphate buffered saline (PBS), buffers, solution or lipid solutions. In all cases, the composition must be sterile and should be fluid to the extent that easy syringability exists. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent that delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, the preferred methods of preparation are vacuum drying and freeze-drying, which yield a powder of the active ingredient plus any additional desired ingredient from a previously sterile-filtered solution thereof.

Oral compositions generally include an inert diluent or an edible carrier. For the purpose of oral therapeutic administration, the active compound can be incorporated with excipients and used in the form of tablets, troches, or capsules, e.g., gelatin capsules. Oral compositions can also be prepared using a fluid carrier for use as a mouthwash. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds can be delivered in the form of an aerosol spray from a pressured container or dispenser that contains a suitable propellant, e.g., a gas such as carbon dioxide, or a nebulizer. Such methods include those described in U.S. Pat. No. 6,468,798.

Systemic administration of a therapeutic compound as described herein can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

In one embodiment, the therapeutic compounds are prepared with carriers that will protect the therapeutic compounds against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Such formulations can be prepared using standard techniques, or obtained commercially, e.g., from Alza Corporation and Nova Pharmaceuticals, Inc.

The pharmaceutical compositions can be included in a container, pack, or dispenser together with instructions for administration.

### EXAMPLES

The invention is further described in the following examples, which do not limit the scope of the invention described in the claims.

### Materials and Methods

The following materials and methods were used in the Examples below.

### Humans

Human AV node and LV tissues were obtained from fully de-identified heart specimens collected during routine autopsy of patients with no known cardiac conduction disease. Tissue sampling was approved by the Partners Healthcare Institutional Review Board under protocol #2015P001827. All patients gave written informed consent.

### Mice

C57BL/6, B6.129P-Cx3cr1^{tm1Litt}/J (Cx₃cr1^{GFP}), B6.129P2(Cg)-Cx3cr1^{tm2.1(cre/ERT)Litt}/WganJ (Cx₃cr1^{CreER}), B6.Cg-Gt(ROSA) 26Sor^{tm32(CAG-COP4*H134R/EYFP)Hze}/J (ChR2^{fl/fl}), B6.129S7-Gja1^{tm1Dlg}/J (Cx43^{fl/fl}), B6; C3Fe a/a-Csf1^{op}/J (Csf1^{op/+}), C57BL/6-Tg(UBC-GFP)30Scha/J (Ubc^{GFP}) and B6.FVB-Tg(ITGAM-DTR/EGFP)34Lan/J (Cd11b^{DTR}) were purchased from Jackson Laboratory. Genotyping for each strain was performed as described on the Jackson Laboratory website. One- to 2-day-old C57BL/6 pups were purchased from Charles River Laboratories. All experiments (except the isolation of neonatal mouse cardiomyocytes) were performed with 8- to 40-week-old animals and were carried out using age- and gender-matched groups. All mice were maintained in a pathogen-free environment of the Massachusetts General Hospital animal facility, and all animal experiments were approved by the Subcommittee on Animal Research Care at Massachusetts General Hospital.

### In Vivo Interventions

Mice were put into parabiosis using either C57BL/6 and Cx₃cr1^{GFP/+} or Cd11b^{DTR} and Cx₃cr1^{GFP/+} mice as described previously (12). Tamoxifen was given as a solution in corn oil (Sigma) to Cx₃cr1^{wt/CreER} ChR2^{wt/fl} or Cx₃cr1^{wt/CreER} Cx43^{fl/fl} mice by intraperitoneal injection. Animals received 5 doses of 2 mg of tamoxifen with a separation of 24 hours between doses. Cx₃cr1^{wt/CreER} ChR2^{wt/fl} and Cx₃cr1^{wt/CreER} Cx43^{fl/fl} mice were analyzed 2 and 7 days post-tamoxifen treatment, respectively. Macrophage depletion was achieved by a single intraperitoneal injection of diphtheria toxin (DT, 25 ng/g body weight) in Cd11b^{DTR} mice (12). C57BL/6 mice injected with DT were used as controls. Clodronate liposomes were kindly provided by Dr. Kory J. Lavine and contained 18 mg of clodronate per mL of liposomes. Depletion studies were performed by intraperitoneal injection of 100 μL/30 g mouse (13).

### EP Study

EP studies were performed under general anaesthesia induced by administering 5% isoflurane driven by an oxygen source into an induction chamber. Anaesthesia was subsequently maintained with 1-2% isoflurane in 95% 02. For EP study, an octapolar catheter (EPR-800) was inserted into the right jugular vein and positioned in the right atrium and ventricle. Programmed electrical stimulation was performed using a standard protocol with 120 ms and 100 ms drive trains and single extrastimuli to measure function of the AV node and the conduction properties of atrial and ventricular tissue. The Wenckebach cycle length was measured by progressively faster atrial pacing rates. Retrograde (VA) conduction cycle length was measured by progressively slower ventricular pacing rates. Sinus node function was determined by measuring the sinus node recovery time (SNRT) following 30 seconds of pacing at three cycle lengths (120, 100 and 80 ms). SNRT was divided by the basic cycle length to adjust for the intrinsic heart rate.

### Ambulatory ECG Telemetry

Continuous ambulatory ECG telemetry was performed by implanting an ETA-F10 transmitter during general anaesthesia with isoflurane. The transmitter was implanted in the abdomen and the leads were tunneled subcutaneously to the upper right and lower left chest resulting in a lead II position. Telemetry data was recorded continuously via a receiver placed under the mouse cage. Data analysis was performed using LabChart Pro software.

### Surface ECG

Mice were anesthetized as described above and surface ECG was recorded using subcutaneous electrodes connected to the Animal Bio amplifier and PowerLab station (AD Instruments). The ECG channel was filtered between 0.3 and 1000 Hz and analyzed using LabChart Pro software. Atropine (1 mg/kg), epinephrine (2 mg/kg) or isoproterenol (20 mg/kg) were administered intravenously, and changes were examined before and after injection.

### Optogenetics

Two days after tamoxifen treatment, Cx₃cr1^{wt/CreER} (control) and Cx₃cr1^{wt/CreER} ChR2^{wt/fl} (Cx₃cr1 ChR2) mice were euthanized and the hearts were perfused in a custom-built, horizontal perfusion bath in Langendorff mode with oxygenized Krebs-Henseleit solution containing (in mM): 118 NaCl, 4.7 KCl, 1.2 MgSO₄, 1.55 CaCl₂, 24.9 NaHCO₃, 1.2 KH₂PO₄, 11.1 Dextrose, pH 7.4 (all Sigma). Recording and electrical pacing electrodes were connected to the heart, and the endocardial surface overlying the AV node was exposed by carefully opening the right atrial free wall above the AV groove. Mean perfusion pressure was maintained at between 60-80 mmHg throughout the experiment and adequacy of the preparation was determined by robust return of sinus rhythm in the perfused heart and visual evidence of vigorous contraction. The location of the AV node was identified grossly under a dissecting microscope. The Wenckebach cycle length was first determined without illumination by determining the electrical stimulation atrial pacing rate at which progressive PR interval prolongation occurred, culminating in a non-conducted atrial impulse due to AV block. The heart was subsequently electrically paced at the determined Wenckebach cycle length and the AV node was subjected to alternating 10-second cycles with and without continuous AV node illumination. Continuous illumination of the exposed AV node was performed using a 400 μm core fiber optic cannula coupled to a 470 nm LED (ThorLabs) at light intensities of 55.7 mW/mm². The recorded ECG tracings were analyzed using LabChart Pro software. The average number of conducted atrial stimuli between two non-conducted impulses during rapid pacing-induced Wenckebach block was determined for each light off and on cycle.

### Tissue Processing

Peripheral blood for flow cytometric analysis was collected by retro-orbital bleeding using heparinized capillary tubes (BD Diagnostics) and red blood cells were lysed with 1× red blood cell lysis buffer (BioLegend). To determine electrolyte levels, blood was collected by cardiac puncture and electrolytes were measured on serum with EasyLyte PLUS analyzer (Medica). For organ harvest, mice were perfused through the LV with 10 mL of ice-cold PBS. Hearts were excised and processed as whole or subjected to AV node microdissection as described previously (32). Briefly, the triangle of Koch, which contains the AV node, was excised by using the following landmarks: ostium of the coronary sinus, tendon of Todaro and septal leaflet of the tricuspid valve. The presence of the AV node was confirmed with HCN4 and acetylcholinesterase staining (see below). After harvest, cardiac tissues were minced into small pieces and subjected to enzymatic digestion with 450 U/mL collagenase I, 125 U/mL collagenase XI, 60 U/mL DNase I, and 60 U/mL hyaluronidase (all Sigma) for 20 minutes (microdissected AV node) or 1 hour (whole heart) at 37° C. under agitation. Tissues were then triturated and cells filtered through a 40 μm nylon mesh (BD Falcon), washed and centrifuged to obtain single-cell suspensions. Peritoneal cells were recovered by lavage with 5 mL of ice-cold PBS supplemented with 3% fetal bovine serum and 2 mM EDTA.

### Flow Cytometry

Isolated cells were first stained at 4° C. in FACS buffer (PBS supplemented with 0.5% bovine serum albumin) with mouse hematopoietic lineage markers including phycoerythrin (PE)- or biotin-conjugated anti-mouse antibodies directed against B220 (1:600), CD49b (1:1200), CD90.2 (1:3000), Ly6G (1:600), NK1.1 (1:600) and Ter119 (1:600). This was followed by a second staining for CX₃CR1 (1:600), CD11b (1:600), CD11c (1:600), CD45 (1:600), CD64 (1:600), CD103 (1:600), CD115 (1:600), F4/80 (BM8, 1:600) Ly6C (1:600) and/or Pacific Orange-conjugated streptavidin. Monocytes were identified as (B220/CD49b/CD90.2/Ly6G/NK1.1/Ter119)^{low} CD11b^{high} CD115^{high} Ly6C^{low/high}. Cardiac macrophages were identified as (B220/CD49b/CD90.2/CD103/Ly6G/NK1.1/Ter119)^{low} (CD45/CD11b)^{high} Ly6C^{low/int} F4/80^{high}. Data were acquired on an LSRII (BD Biosciences) and analyzed with FlowJo software.

### Cell Sorting

To isolate peritoneal macrophages, depletion of undesired cells including lymphocytes was performed using MACS depletion columns according to the manufacturer's instructions (Miltenyi). Briefly, single cell suspensions after peritoneal lavage were stained using a cocktail of PE-conjugated antibodies directed against B220, CD49b, CD90.2, NK1.1 and Ter119, followed by incubation with anti-PE microbeads. The enrichment of peritoneal macrophages was evaluated by flow cytometry. To purify macrophages from AV node tissue, digested samples were stained with hematopoietic lineage markers, CD11b, CD45, F4/80 and Ly6C, and macrophages were FACS-sorted using a FACSAria II cell sorter (BD Biosystems). 4′,6-diamidino-2-phenylindole (DAPI, Thermo Fisher Scientific) was used as a cell viability marker. To isolate cardiac macrophages from whole heart, digested tissue samples were first enriched for CD11b⁺ cells using CD11b microbeads and MACS columns according to the manufacturer's instructions. Next, cells were stained with hematopoietic lineage markers, CD45, F4/80 and Ly6C, and FACS-sorted using a FACSAria II cell sorter.

### Isolation and Culture of Neonatal Mouse Cardiomyocytes

Neonatal mouse cardiomyocytes were isolated by use of enzymatic dissociation. One- to 2-day-old pups were sacrificed, the hearts removed and the ventricles harvested. The tissue was dissociated in HBSS containing 0.1% trypsin (Sigma) overnight at 4° C. under agitation, followed by three consecutive digestion steps in HBSS containing 335 U/mL collagenase II (Worthington Biochemical Corporation) for 2 minutes at 37° C. with gentle agitation. The digest was filtered through a 40 μm nylon mesh, washed and resuspended in mouse culture medium which consisted of DMEM supplemented with 14% FBS and 2% penicillin/streptomycin. Cell suspensions were preplated into 100 mm cell tissue culture dishes and incubated at 37° C. for 45 minutes to allow preferential attachment of non-myocyte cell populations and enrichment of the cardiomyocyte population. Cardiac cells remaining in suspension were collected and seeded at a density of 0.5-1×10⁵ cells/cm² on fibronectin-coated 8 mm cover slips (Warner Instruments) pre-seeded with 5×10⁴ FACS-purified GFP⁺ cardiac macrophages. Medium exchanges were performed on the first day after seeding and every other day thereafter with mouse culture medium supplemented with 1 μM cytosine β-D-arabinofuranoside hydrochloride (Sigma). Experiments were performed on day 3.

### Whole-Cell Patch Clamp

Membrane potentials were recorded with whole-cell patch clamp technique in tight-seql current-clamp mode at 37° C. Borosilicate-glass electrodes filled with pipette solution had 4 to 6 MΩ tip resistance, and were connected with an Axopatch 200B amplifier and a Digidata 1440A A/D converter. Data were analyzed with Clampfit. The bath solution contained (in mM): 136 NaCl, 5.4 KCl, 1 MgCl₂, 1.8 CaCl₂, 0.33 NaH₂PO₄, 5 HEPES, 10 Dextrose, pH 7.4 with NaOH, and the pipette solution contained (in mM): 110 K-aspartate, 20 KCl, 1 MgCl₂, 5 MgATP, 0.1 GTP, 10 HEPES, 5 Na-Phosphocreatine, 0.05 EGTA, pH 7.3 with KOH (all Sigma). To identify the patched cell, the pipette was additionally loaded with 0.2 mg/mL Texas Red⁺ dextran (MW 3000). To block Cx43-mediated gap junction communication, 200 μM of the Cx43-mimetic peptide Gap26 was added to the batch solution during patch clamp recording.

### Voltage Dye Imaging

Cardiomyocyte-macrophage co-cultures were loaded with 4 μM of ANNINE-6plus for 5 minutes in Tyrode's solution containing (in mM): 140 NaCl, 5.4 KCl, 1.8 CaCl₂, 1 MgCl₂, 10 glucose and 10 HEPES, pH 7.4 with NaOH (all Sigma). After washing, cover slips were transferred to Tyrode's solution containing 20 μM of blebbistatin to uncouple the excitation-contraction process in cardiomyocytes. To optically detect action potentials, line scans were obtained from the surface membranes of cardiomyocytes and attached macrophages using an Olympus IV100 microscope. The acquired line-scans were filtered with a collaborative filter to increase the signal-to-noise ratio and analyzed in Matlab as previously described (34). In detail, the average signal intensity of each successive line in the line-scan image corresponding to the membrane of the cell of interest was calculated to obtain the time course of the averaged fluorescence [F(t)]. The time course of normalized fractional fluorescence changes [ΔF/F₀(t)], where ΔF is F(t)-F₀(t) and F₀(t) is the baseline trace, was subsequently determined for the cardiomyocyte and attached macrophage.

### Immunofluorescence Staining

To eliminate blood contamination, hearts were perfused with 10 mL of ice-cold PBS. Hearts from Cx₃cr1 ChR2, Cx₃cr1 Cx43^{-/-}, Csf1^{op} and Cd11b^{DTR} mice were embedded in OCT compound and flash-frozen in a 2-methylbutane bath on dry ice. Serial frozen 6 to 25 μm sections were prepared and acetylcholinesterase staining was carried out to identify the AV node. The selected sections were fixed with 10% formalin for 5 minutes, washed and permeabilized with 0.1% Triton X-100 in PBS for 30 minutes. The tissue sections were then blocked with 4% normal goat serum in PBS for 30 minutes at room temperature. After blocking, sections were incubated with a rabbit anti-mouse HCN4 antibody (Alomone Labs) overnight at 4° C., followed by a biotinylated goat anti-rabbit IgG antibody for 45 minutes and DyLight 649-streptavidin for 30 minutes at room temperature. The sections from Cx₃cr1 ChR2 hearts were additionally incubated with a chicken anti-GFP antibody overnight at 4° C. Alexa Fluor 568 goat anti-chicken IgY antibody was used as a secondary antibody. The sections from Cx₃cr1 Cx43^{-/-} and Csf1^{op} hearts were additionally incubated with a rat anti-mouse CD68 antibody for 2 hours at room temperature. Alexa Fluor 568 goat anti-rat IgG antibody was used as a secondary antibody. TUNEL staining was performed using DeadEnd Fluorometric TUNEL system according to the manufacturer's protocol and DAPI was applied for nuclear counterstaining. Cover slips seeded with cardiomyocytes and GFP⁺ FACS-purified cardiac macrophages were fixed with 4% PFA for 10 minutes at room temperature. After washing, cells were permeabilized with 0.1% Triton X-100 in PBS for 10 minutes at room temperature, washed and blocked in blocking solution (PBS containing 10% goat serum, 0.1% Tween-20 and 0.3 M glycine) for 1 hour at room temperature. Cells were then stained with rabbit anti-mouse Cx43 antibody in blocking solution for 1 hour at room temperature, followed by incubation with Alexa Fluor 647 goat anti-rabbit IgG secondary antibody for 1 hour at room temperature. After washing, cells were stained with Alexa Fluor 568 anti-Desmin antibody and DAPI was applied for nuclear counterstaining. All images were captured using an Olympus FV1000 or a Nikon 80i fluorescence microscope and processed with ImageJ software.

### Whole-Mount Immunofluorescence Staining

AV nodes from Cx₃cr1^{GFP/+} mice were harvested as described above and fixed using periodate-lysine-paraformaldehyde (PLP) in a 96-well plate for 1 hour at room temperature. Tissues were washed in PBS, and processed as whole or embedded in 4% agarose and cut in 300 μm sections using a Pelco 101 vibratome. Tissues were then washed in 1% Triton X-100 diluted in PBS, and blocked and permeabilized in blocking solution (PBS containing 20% goat serum, 1% Triton X-100 and 0.2% sodium azide) for 1 hour at room temperature. AV nodes were then stained with chicken anti-GFP, rabbit anti-mouse Cx43 and rat anti-mouse HCN4 (Abcam) antibodies in blocking solution for 3 days at 4° C. After washing, samples were incubated with Alexa Fluor 488 goat anti-chicken IgY, Alexa Fluor 568 goat anti-rabbit IgG and Alexa Fluor 647 goat anti-rat IgG secondary antibodies overnight at 4° C. For fibroblast quantification, sections were incubated with PDGFRα-APC antibody overnight at 4° C. and DAPI was applied for nuclear counterstaining. AV nodes were then optically cleared or mounted between two long coverslips and imaged using an Olympus FV1000 microscope and z-stack images acquired at 0.1 to 2 μm steps were processed with ImageJ software. Human AV node and LV tissues were fixed using 4% PFA for 24 hours at 4° C. Tissues were washed in PBS, embedded in 4% agarose and 500 μm sections were cut using a Pelco 101 vibratome. The sections were then washed in PBS containing 2% Triton X-100 and 20% DMSO, followed by blocking and permeabilization in blocking solution (PBS containing 20% goat serum, 2% Triton X-100, 20% DMSO and 0.2% sodium azide) for 1 hour at room temperature. Tissue sections were stained with mouse anti-human CD68 (clone EBM11) or mouse anti-human CD163 and rabbit anti-human Cx43 antibodies in blocking solution for 7 days at 4° C. After washing, samples were incubated with Alexa Fluor 488 goat anti-mouse IgG and Alexa Fluor 568 goat anti-rabbit IgG secondary antibodies for 7 days at 4° C. Stained human tissue sections were then washed, optically cleared and imaged.

### Optical Clearing

Mouse and human tissues were cleared using Rapiclear 1.49 by immersion in the clearing solution for 24 hours at room temperature. The cleared tissues were then mounted on a custom-made sample holder and imaged using an Olympus FV1000 microscope. Acquired images were processed with Amira 3D software.

### Immunohistochemistry

Human AV node samples were stained with Masson's Trichrome to identify the cardiac conduction tissue. To identify human cardiac macrophages, the paraffin-embedded tissue was first deparaffinized and antigen retrieval was performed using sodium citrate, pH 6.0 (BD Biosciences). In order to block endogenous peroxidase activity, the tissue sections were incubated in 1% H₂O₂ diluted in dH₂O for 10 minutes and rinsed in dH₂O and PBS. The sections were then blocked with 4% horse serum in PBS for 30 minutes at room temperature and incubated with a monoclonal mouse anti-human CD68 antibody (clone: KP1) overnight at 4° C. A biotinylated horse anti-mouse IgG antibody was applied for 30 minutes at room temperature. For color development, the VectaStain ABC kit and AEC substrate were used. All the slides were counterstained with Harris hematoxylin and scanned with NanoZoomer 2.0-RS (Hamamatsu). Sections were analyzed at 20× magnification using iVision software.

### Electron Microscopy

Hearts from Cx₃cr1^{GFP/+} mice were fixed using PLP solution and frozen 50 μm sections were incubated in 0.3% H₂O₂ diluted in PBS for 10 minutes, followed by incubation with PBS containing 1% BSA and 0.05% saponin for 1 hour at room temperature. A rabbit anti-GFP antibody was applied to the sections and incubated overnight at 4° C. The tissue sections were washed and incubated with a biotinylated goat anti-rabbit IgG antibody for 2 hours at room temperature. After washing, sections were incubated with VectaStain ABC reagent for 30 minutes at room temperature, washed and then fixed with PBS containing 1% glutaraldehyde and 5% sucrose for 30 minutes at room temperature. For color development, diaminobenzidine solution was applied followed by 1% H₂O₂ in dH₂O. The sections were washed and incubated with 1% osmium tetroxide in 0.1 M sodium cacodylate buffer on ice for 30 minutes. Prior to embedding, sections were dehydrated and allowed to pre-infiltrate in a 1:1 mix of Eponate resin and propylene oxide overnight at room temperature with gentle agitation. Sections were then infiltrated with fresh 100% Eponate resin and polymerized for 1-2 days at 60° C. Polymerized sections were trimmed and oriented such that the targeted AV node region would lie at the sectioning face. Thin sections were cut using a Leica EM UC7 ultramicrotome, collected onto formvar-coated grids, stained with uranyl acetate and Reynold's lead citrate and examined in a JEOL JEM 1011 transmission electron microscope at 80 kV. Images were collected using an AMT digital imaging system (Advanced Microscopy Techniques).

### YFP Target-to-Background Ratio (TBR) Measurement

Cx₃cr1^{wt/wt} and Cx₃cr1^{wt/CreER} mice were intravenously injected with 4 μg of CX₃CR1-PE and Sca1-APC antibodies to label tissue-resident macrophages and endothelial cells, respectively. After 30 minutes of in vivo labeling, mice were perfused through the LV with 10 mL of ice-cold PBS. Hearts were then mounted between two long coverslips and imaged using an Olympus IV100 microscope. Z-stack images acquired at 1 μm steps were analyzed in Matlab with custom developed functions. Semi-automatic thresholding-based algorithms were used for TBR measurements. A BM3D filter method was implemented for noise reduction to increase the overall signal-to-noise ratio.

### Western Blot

Total protein was extracted from heart tissue in RIPA lysis buffer supplemented with protease/phosphatase inhibitor cocktail. Protein concentration was measured using BCA assay. Lysates of 3 μg were then subjected to electrophoresis using NuPAGE Novex Gel system (Thermo Fisher Scientific) and were blotted to nitrocellulose membrane using iBlot Gel Transfer system (Thermo Fisher Scientific) according to manufacturer's instructions. Anti-mouse Cx43 antibody, anti-mouse GAPDH antibody and HRP-coupled secondary antibodies were used. Signals were visualized with chemiluminescent substrate and densitometric analysis was performed with ImageJ.

### PCR Confirmation of the Deletion of the Cx43 Allele

Genomic DNA from FACS-purified cardiac macrophages was isolated with DNeasy Blood & Tissue kit and used in PCR with two pairs of Cx43-specific primers for detecting Cx43^{fl} or Cx43^{wt} alleles, and for detecting the Cx43 allele lacking the floxed fragment. To normalize the amount of input DNA, specific primers to the Cx₃cr1^{wt} gene were used.

qPCR

Total RNA from whole AV node tissue was extracted using the RNeasy Micro kit or from FACS-purified cells using the PicoPure RNA isolation kit according to the manufacturer's protocol. First-strand cDNA was synthesized using the High-Capacity RNA-to-cDNA kit and pre-amplified using the TaqMan PreAmp Master Mix kit according to the manufacturer's instructions. TaqMan gene expression assays were used to quantify target genes. The relative changes were normalized to Gapdh mRNA using the 2^{-ΔΔCT} method.

### Bulk RNA-Seq

Total RNA from whole AV node tissue was extracted using the RNeasy Micro kit according to the manufacturer's protocol. The RNA quality was assessed with the RNA 6000 Pico assay kit using the Agilent Bioanalyzer. Sequencing-ready cDNA libraries were prepared using the NEBNext Ultra RNA Directional Library Prep kit for Illumina following the manufacturer's protocol. Bioanalyzer traces were used to confirm library size distribution. The libraries were quantified by qPCR using KAPA Library Quantification kit and then sequenced as single-end 50 base reads on a Illumina HiSeq 2000 in high-output mode.

### Single-Cell RNA-Seq

AV node macrophages were FACS-purified from whole AV node tissue as described above. Single macrophages were then captured using the Fluidigm C1 microfluidic chip designed for 5 to 10 μm cells according to the manufacturer's protocol. A concentration of 1.8×10⁵ cells per mL was used for chip loading. After cell capture, chips were examined visually to identify empty chambers, which were excluded from later analysis. Cell lysis and cDNA synthesis were performed on-chip with SMARTer Ultra Low RNA kit for the Fluidigm C1 system. Amplified cDNA was validated and quantified on an Agilent Bioanalyzer with the High Sensitivity DNA chip. Illumina libraries were then constructed in 96-well plates using the Nextera XT DNA Sample Preparation kit according to a modified protocol supplied by Fluidigm. Constructed libraries were validated and quantified with the High Sensitivity DNA chip, and subsequently normalized and pooled to equal concentrations. The pooled libraries were quantified by qPCR and sequenced as single-end 50 base reads on a Illumina HiSeq 2000 in high-output mode.

### Bulk RNA-Seq

Transcriptome mapping was performed with STAR v2.3.0 (35) using the Ensembl 67 release exon/splice-junction annotations. Approximately 65-78% of reads mapped uniquely. Read counts for individual genes were calculated using the unstranded count feature in HTSeq v0.6.0 (36). Differential expression analysis was performed using the exactTest routine of the edgeR R package (37) after normalizing read counts and including only those genes with counts per million (cpm)>1 for two or more replicates. Differentially expressed genes were then defined as those genes with >2-fold change in expression and false discovery rate (FDR)<0.05. Hierarchical clustering of differentially expressed genes was performed with the heatmap.2 function in the R gplots library. Gene Set Enrichment Analysis (GSEA) was performed as described previously (38). Input rankings were based on the sign of the fold change multiplied by the inverse of the p value. Genes involved in cardiac conduction (gene ontology term GO:0061337, 38 unique members) were downloaded from the QuickGO Browser (www.ebi.ac.uk/QuickGO/).

### Single-Cell RNA-Seq

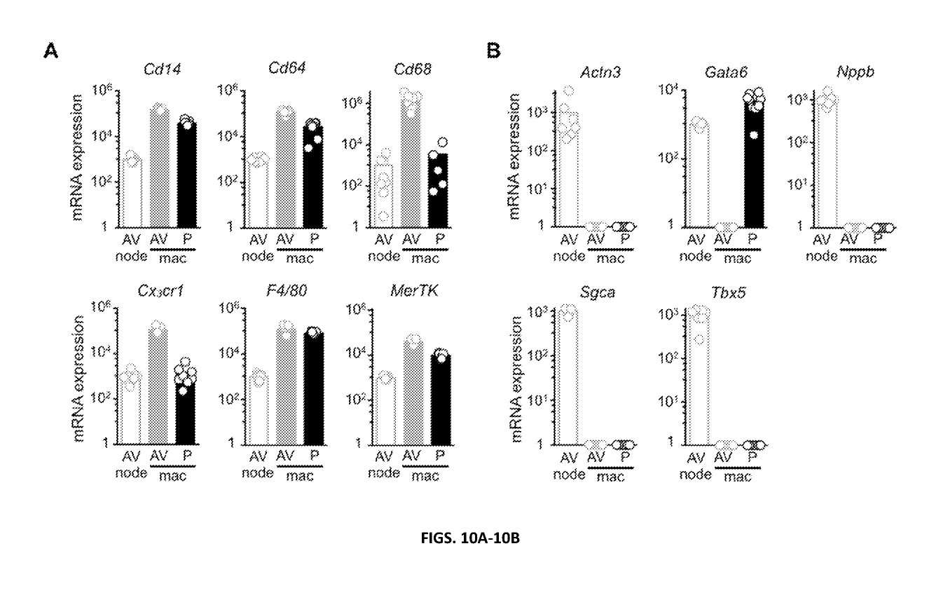

Transcriptome mapping (73-87% reads were uniquely mapped) and counts per gene calculations were performed in the same manner as with the bulk RNA-seq data. The 76 cells with the most reads (260K-6.3M, median 2.1M) were selected for further analysis. Expression thresholding for detected genes and calculation of overdispersion (i.e., higher than expected variance) was performed with SCDE (39) using the clean.counts and pagoda.varnorm routines, respectively, which resulted in 9,235 genes retained for further analysis. Hierarchical clustering of the 200 most overdispersed genes was performed using the heatmap.2 function in the R gplots library. To group cells into three co-expression categories based on H2 and Ccr2 expression levels, we performed spectral clustering on their joint distribution based on log 2(cpm) values (specc command in the factoextra R library). Then, the two clusters with lowest average H2 expression were joined to form a larger cluster shown in orange in FIG. 9A.

### Microarray

Raw microarray data from (11) were downloaded from ArrayExpress (www.ebi.ac.uk/arrayexpress), accession number E-MEXP-3347, and normalized using the robust multi-array average (33). GSEA was performed using standard parameters (gene set permutation, signal-to-noise ratio as a ranking metric).

### Computational Modeling

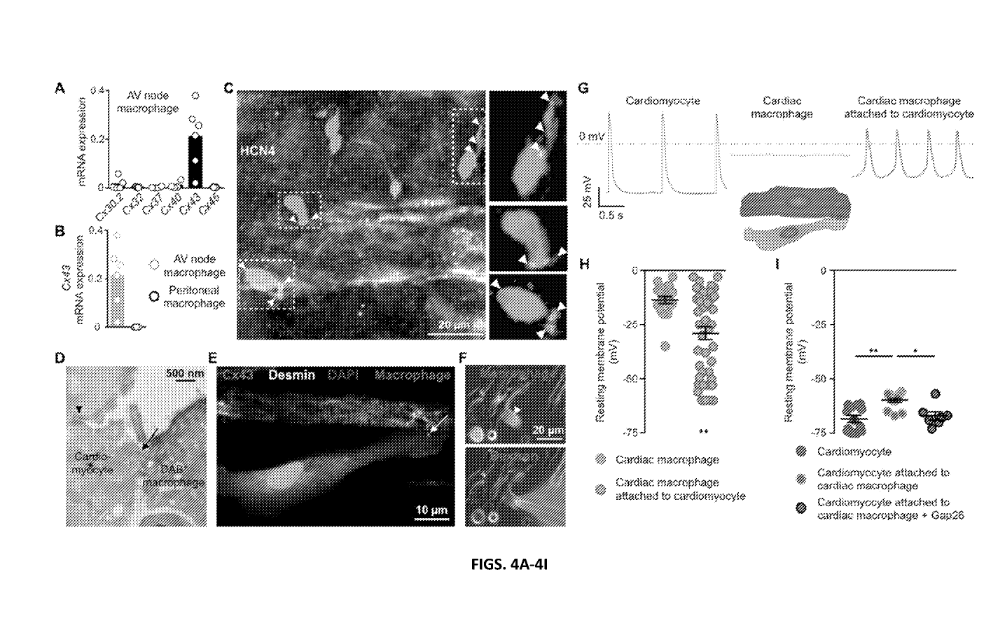

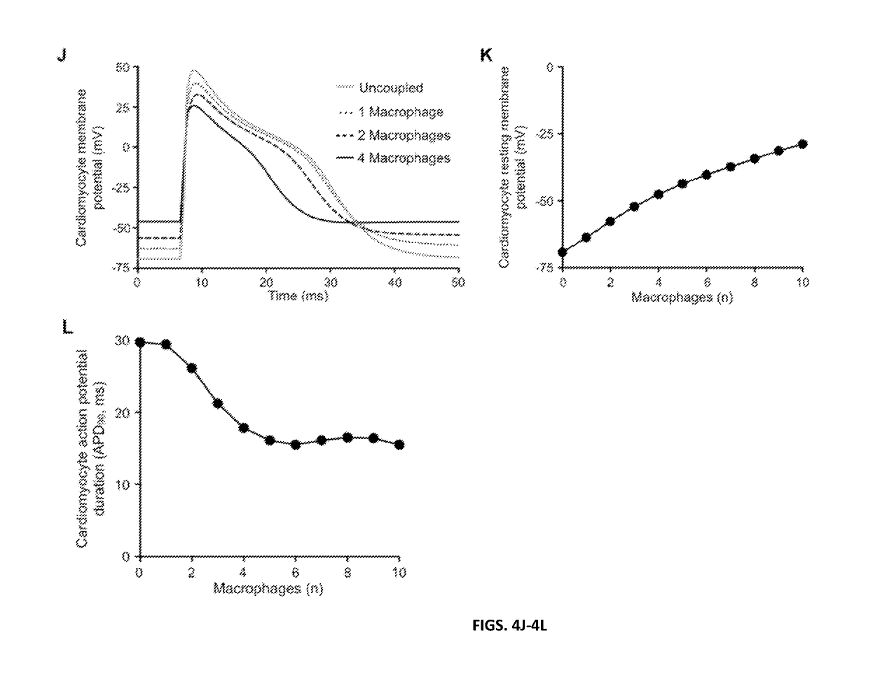

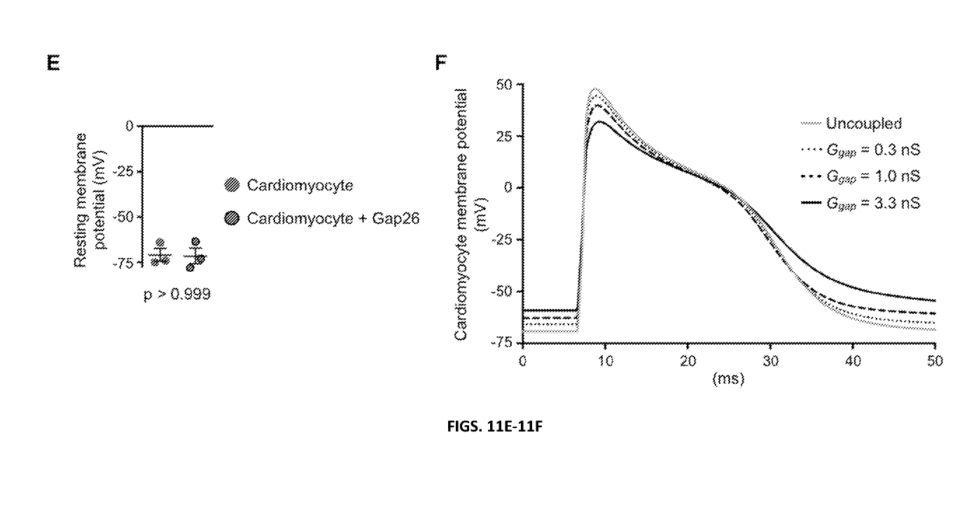

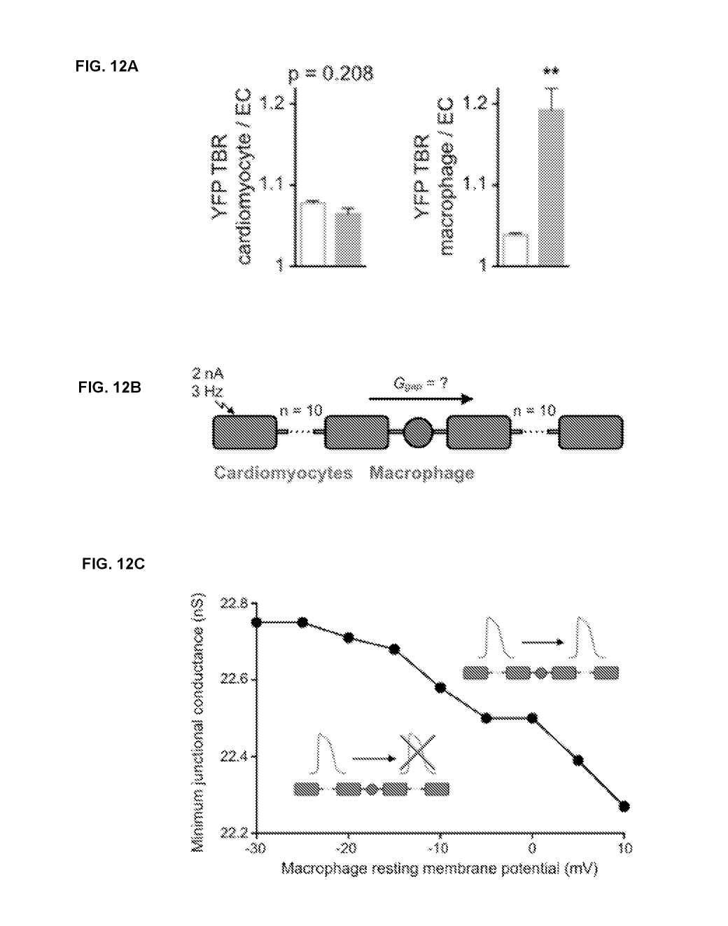

Macrophages were modeled as unexcitable cells based on a previously published model (40), which was adjusted using the experimental whole-cell patch clamp data recorded for cardiac macrophages in this study (FIGS. 4G, 4H, 11A and 11B). The resulting macrophage model comprises an inwardly rectifying potassium current and an unspecific background current. Table 1 shows the constants of the model. Potassium concentrations were set to match experimental conditions. The remaining parameters Cₘ, G_{b}, and G_{Kir} were fitted to the experimental whole-cell patch clamp data. The membrane capacitance of the model, Cₘ, was set to the mean of the measured macrophage membrane capacitances (n=18). The conductance of the unspecific background current, G_{b}, was set to the inverse of the mean of measured membrane resistances (n=9). Finally, the maximal conductance of the potassium channel, G_{Kir}, was adapted such that the resulting resting membrane potential matched the measurements (n=20). The resulting resting membrane potential also served as initial value for the membrane potential Vₘ of the model. A mathematical model of a rabbit AV bundle cardiomyocyte (41) was adapted to mouse cells to be able to estimate the effects of macrophage coupling to an AV bundle cardiomyocyte. The rabbit model was modified such that the action potential duration (APD₉₀) was reduced from 48 ms to 30 ms, a physiological value for mouse atrial cardiomyocytes (42). For this purpose, we introduced two scaling factors for the time constants of gating variables that correspond to the currents I_{Ca,L}, and Iₜₒ. Namely, in the altered model it is τ_{*}=s_{*}τ̅_̅{̅*̅}̅ for *∈{d, r, pᵢ} where τ̅_̅{̅*̅}̅ is the corresponding original value from the unaltered model. The resulting scaling factors of the modified model were s_{d}=0.5182 and sᵣ=7.0239.

TABLE 1
Macrophage Model Constants

| | | |
|---|---|---|
| Parameter Name | Symbol | Value |

| | | | |
|---|---|---|---|
| Temperature | T | 295 | K |
| Intracellular potassium concentration | [K]ₒ | 5.4 | mM |
| Extracellular potassium concentration | [K]ᵢ | 139 | mM |

| Potassium channel parameter | a_{Kir} | 0.94 | |
|---|---|---|---|
| Potassium channel parameter | b_{Kir} | 1.26 | |
| Background current reversal potential | E_{b} | 0 | mV |
| Membrane capacitance | Cₘ | 27.9 | pF |
| Background current conductance | G_{b} | 2.05 | nS |
| Potassium channel maximum conductance | G_{Kir} | 5.23 | nS |
| Initial membrane potential | Vₘ | −13.6 | mV |

### Quantification and Statistical Analysis

All statistical analyses were conducted with GraphPad Prism software. Statistical parameters including the exact value of n, the definition of center, dispersion and precision measures (mean±SEM) and statistical significance are reported in the text, FIGS. and Figure Legends. The data was tested for normality using the D'Agostino-Pearson normality test and for equal variance. Statistical significance was assessed by the two-sided Student's t test for normally distributed data. If normal distribution or equal variance assumptions were not valid, statistical significance was evaluated using the two-sided Mann-Whitney test and the two-sided Wilcoxon rank-sum test. For multiple comparisons, nonparametric Kruskal-Wallis tests followed by Dunn's posttest were performed. The Mantel-Cox test was used to compare onset of AV block in DT-treated mice. P values of 0.05 or less were considered to denote significance. Animal group sizes were as low as possible and empirically chosen. No statistical methods were used to predetermine sample size and animals were randomly assigned to treatment groups.

### Data Resources

The transcriptome sequencing data for whole AV node tissues and all single cells have been deposited in the Gene Expression Omnibus database under accession numbers GSE86306 and GSE86310, respectively.

### Example 1: Macrophages Abound in the AV Node

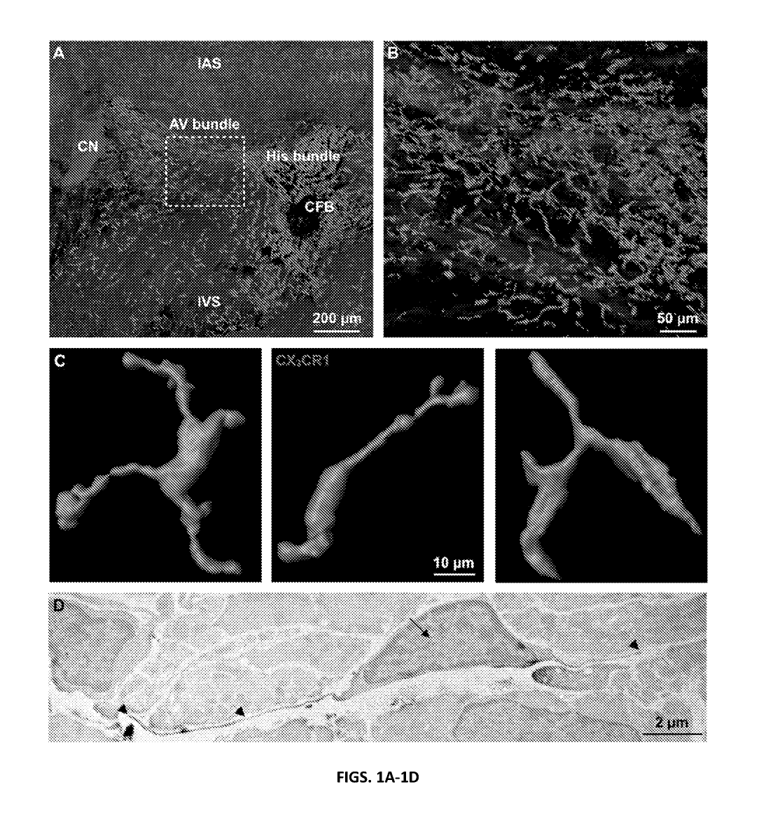

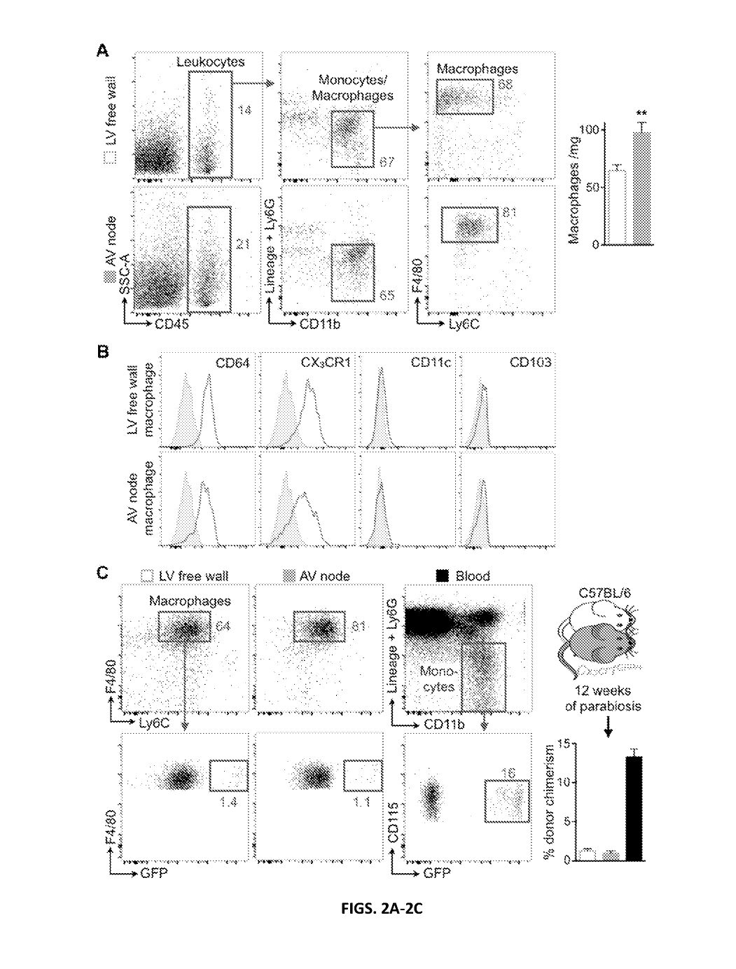

Resident macrophages are present in the left ventricle (LV), but prior work did not report on intra-organ heterogeneity. It therefore remained unclear whether macrophages distribute homogeneously throughout the heart and whether any reside in the conduction system. To investigate macrophages' presence and spatial distribution in the intact AV node, the entire AV nodes of Cx₃cr1^{GFP/+} mice were optically cleared and imaged. Cx₃cr1^{GFP/+} mice are an extensively validated reporter mouse in which green fluorescent protein identifies cardiac macrophages, by confocal microscopy (FIG. 1A). It was found that HCN4-expressing cardiomyocytes, in particular in the lower nodal or AV bundle, frequently intersperse with macrophages (FIG. 1B). AV node macrophages assume an elongated, spindle-shaped appearance with far-reaching cytoplasmic projections (FIG. 1C). To study the morphological characteristics of AV node macrophages by electron microscopy, GFP⁺ macrophages in Cx₃cr1^{GFP/+} mice were labeled with diaminobenzidine (DAB). DAB⁺ macrophages display long cellular processes that closely associate with cardiomyocytes (FIG. 1D).

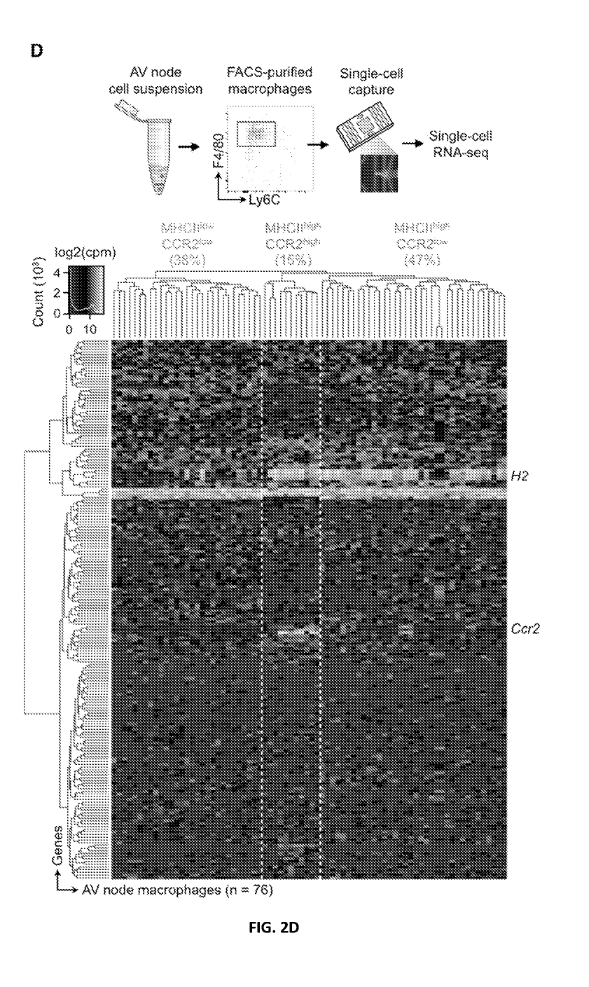

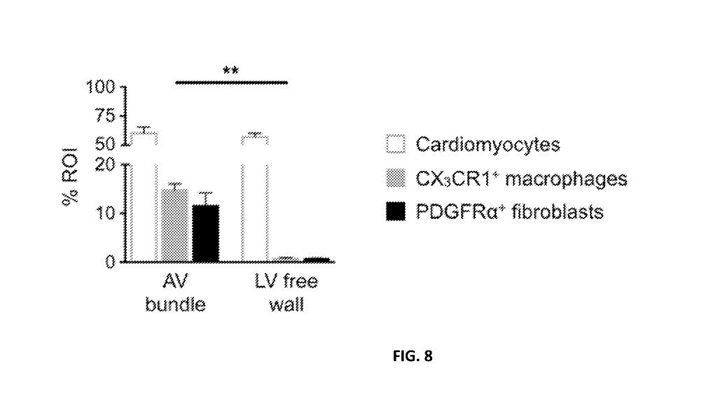

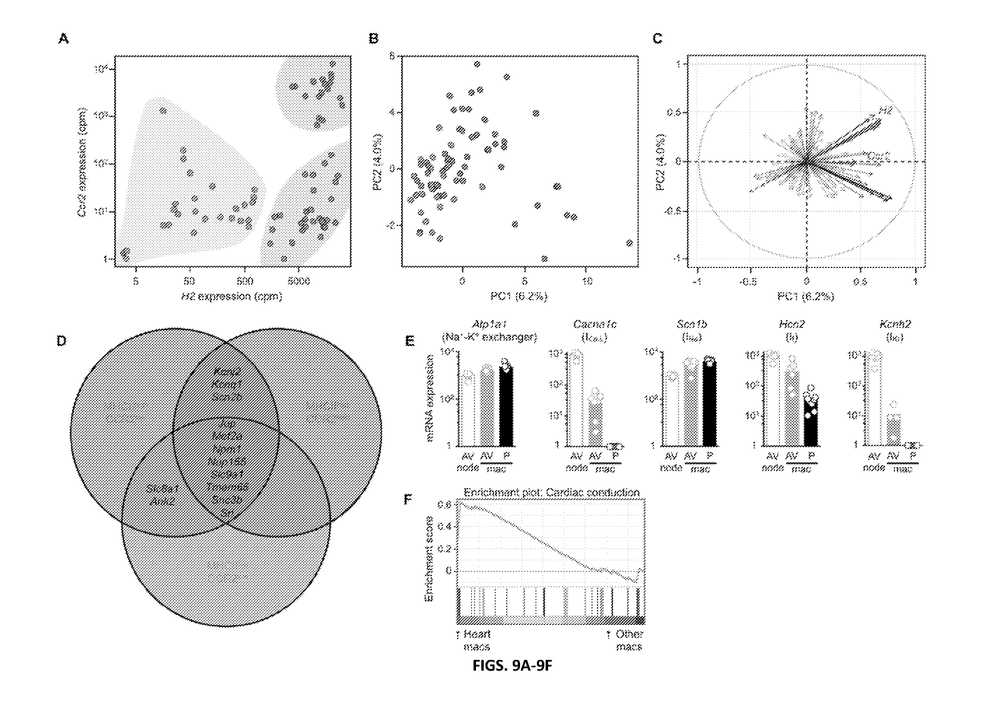

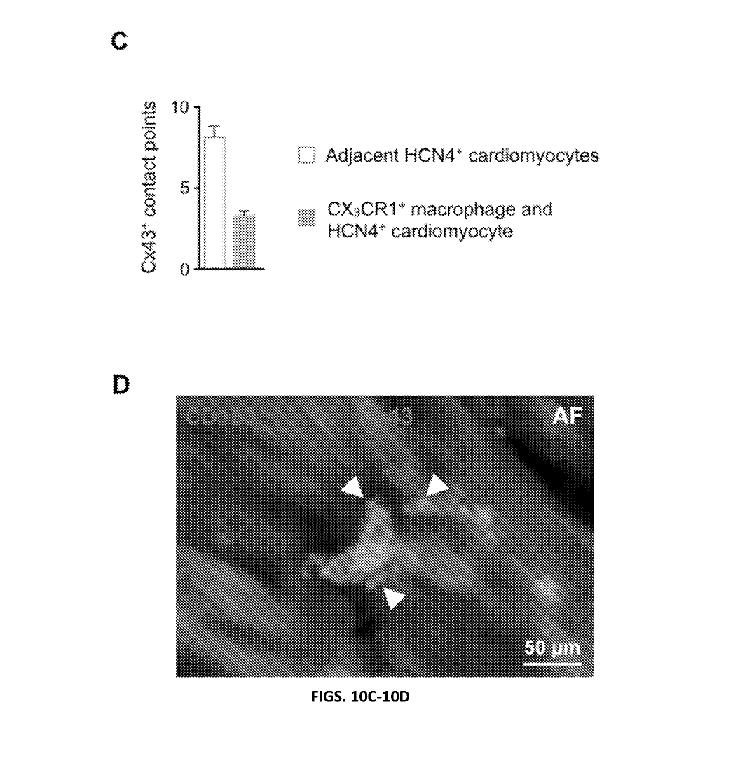

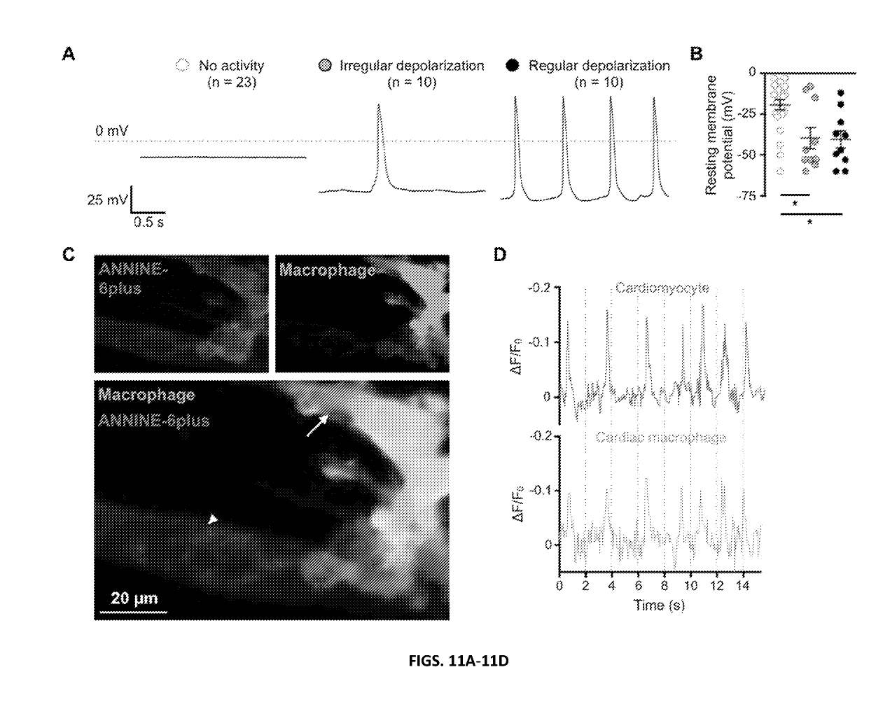

To compare macrophage numbers in the AV node with the LV myocardium, microdissected tissue was examined by flow cytometry and histology. The mouse AV node has a higher macrophage density than the LV (FIGS. 2A and 8). In the mouse AV node, the majority of CD45⁺ leukocytes are CD11b⁺ F4/80⁺ Ly6C^{low} macrophages. Co-expression of CD64 and CX₃CR1 and the lack of CD11c and CD103 expression confirm that these cells are macrophages and not dendritic cells (FIG. 2B). AV node leukocytes display the characteristic core macrophage gene signature suggested by the Immunological Genome Project (FIG. 10A). Furthermore, CX₃CR1⁺ macrophages do not express the fibroblast marker PDGFRα. Taken together, these data suggest that the cells are indeed macrophages, and confirm that Cx₃cr1^{GFP/+} mice are an appropriate strain to study macrophages in the AV node.

Steady-state myocardial tissue-resident macrophages primarily arise from embryonic yolk-sac progenitors and perpetuate independently of monocytes through in situ proliferation. Using parabiosis, it was determined that circulating cells contributed minimally to AV node macrophages, similar to LV free wall macrophages (FIG. 2C).

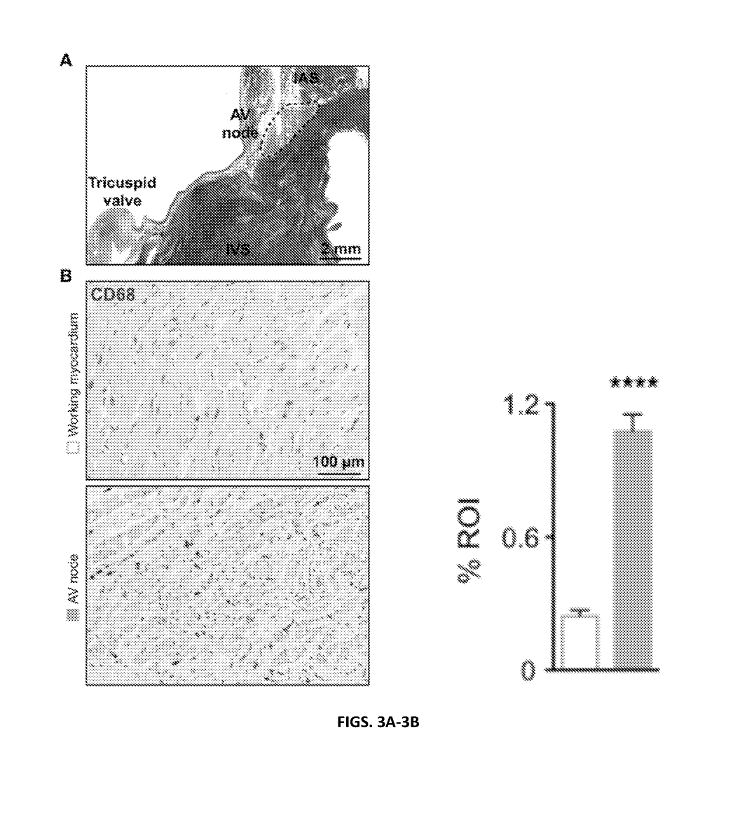

Macrophages in six human AV nodes were also studied. This included optical clearing of AV nodes from autopsy cases. These patients did not die of cardiovascular disease. Fresh AV nodes were harvested within 24 hours after death and underwent optical clearing after staining with the well-validated human macrophage markers CD68 and CD163. Confocal microscopy of 500 μm thick tissue slabs revealed that, in analogy to mice, macrophages were more abundant in human AV nodes than in working myocardium (FIGS. 3A-3B). Human AV node macrophages also exhibit a spindle-shaped appearance with long-reaching protrusions.

Single-cell RNA-sequencing (RNA-seq) of mouse AV node macrophages isolated by flow sorting showed cellular subsets that are also present elsewhere in the heart (FIG. 2D). These macrophage subsets separated based on their expression of major histocompatibility complex class II (H2) and chemokine receptor 2 (Ccr2) (FIGS. 9A-9C). RNA-seq and quantitative real-time PCR (qPCR) revealed that AV node macrophages express ion channels and exchangers (FIGS. 9D and 9E), while deposited microarray data show cardiac macrophages' enrichment of genes associated with conduction (FIG. 9F). Thus, murine AV node macrophages have a similar expression profile as cardiac resident macrophages, including genes involved in electrical conduction.

### Example 2: Connexin 43 Connects Macrophages with Myocytes

Gap junctions, which are formed by connexin (Cx) proteins, connect the cytoplasm of two adjacent cells to enable their communication (15). Most tissues as well as immune cells express Cx43. Cx43-containing gap junctions electrically couple cardiomyocytes, enable electrical impulse propagation, and consequently coordinate synchronous heart muscle contractions. In addition, Cx43-containing gap junctions couple cardiomyocytes with non-cardiomyocytes, which can thereby alter the electrophysiological properties of cardiomyocytes.

To determine if AV node macrophages express proteins that give rise to gap junctions, six connexins found in leukocytes in FACS-purified cells harvested from microdissected AV nodes were evaluated. AV node macrophages mainly express Cx43 (FIG. 4A). Macrophages were sorted from the peritoneal cavity and compared their Cx43 levels with AV node macrophages. AV node macrophages express Cx43 at much higher levels than peritoneal macrophages (FIG. 4B). To ensure the purity and identity of sorted macrophage populations, different macrophage- and cardiomyocyte-specific markers were measured in FACS-purified macrophage populations. All macrophage samples display a characteristic macrophage signature, including Cd14, Cd64, Cd68, Cx₃cr1, F4/80 and MerTK (FIG. 10A), and lack expression of cardiomyocyte-specific genes (FIG. 10B). As reported previously, peritoneal macrophages express Gata6 (16) but AV node macrophages do not (FIG. 10B).

The Cx43 protein expression in AV node macrophages was analyzed by whole-mount immunofluorescence in the lower AV node, an area in which conducting cells express this connexin. Cx43 marks on average three punctate contact points between CX₃CR1⁺ macrophages and HCN4⁺ cardiomyocytes, suggesting gap junction-mediated intercellular communication between both cell types in the distal AV node (FIGS. 4C and 10C). Likewise, the human AV bundle shows punctate Cx43⁺ gap junctions between CD163⁺ macrophages and conducting cardiomyocytes (FIG. 10D). Electron microscopy also visualized direct membrane-membrane contact between AV node macrophages and conducting cardiomyocytes (FIG. 4D). Together, these observations indicate the presence of gap junctions between conducting cells and AV node macrophages.

### Example 3: Macrophages Electrically Modulate Myocytes

Since gap junctions electrotonically couple neighboring cells (17), the hypothesis that macrophages enter electrotonic communication with adjacent cardiomyocytes was tested. The membrane potential of FACS-purified cardiac macrophages attached to neonatal mouse cardiomyocytes was investigated using whole-cell patch clamp. As observed in vivo, Cx43 localized at sites of macrophage-cardiomyocyte interaction, suggesting gap junction communication between these cell types in culture (FIG. 4E). TexasRed⁺ dextran entering GFP⁺ macrophages from the micropipette (FIG. 4F) confirms that the membrane potential recording derived from macrophages. Spontaneously-beating cardiomyocytes displayed a typical resting membrane and action potential (18) (FIG. 4G). The resting membrane potential in solitary cardiac macrophages is depolarized relative to that of cardiomyocytes (FIG. 4G). The documented values between −35 and −3 mV correspond well with data reported for human monocyte-derived and mouse peritoneal macrophages (19) (FIG. 4H). There was no spontaneous depolarization in solitary cardiac macrophages (FIG. 4G). The membrane potential in macrophages attached to beating cardiomyocytes after co-culture of FACS-purified cardiac macrophages with neonatal mouse cardiomyocytes for three days was recorded. 23% of these macrophages rhythmically depolarized with a distinct action potential morphology, characterized by a slowed upstroke and reduced maximal polarization when compared to cardiomyocytes (FIG. 4G). These cardiomyocyte-linked macrophages' resting membrane potentials were more negative than those of solitary macrophages, documenting electrical coupling (FIG. 4H). We recorded irregular depolarization in another 23% of co-cultured macrophages and lack of activity in the remaining 54% (FIG. 11A). Macrophages with any kind of depolarization, either regular or irregular, had a more negative resting membrane potential than non-depolarizing macrophages (FIG. 11B). To simultaneously record action potential-related fluorescence changes in macrophages and cardiomyocytes, cardiomyocyte-driven macrophage depolarization was examined by using the ANNINE-6plus voltage-sensitive dye. These data show that macrophage action potentials are synchronous with action potentials of coupled cardiomyocytes (FIGS. 11C and 11D).

To address the question whether cardiac macrophages are passive bystanders or whether they influence conduction, experiments were performed to investigate whether macrophages change the electrical properties of coupled cardiomyocytes. Indeed, macrophages render cardiomyocyte resting membrane potentials more positive, an effect that was reversed by pharmacological Cx43 blockade (FIG. 4I). In control experiments, inhibition of Cx43-mediated gap junctions in solitary cardiomyocytes did not change their resting membrane potential (FIG. 11E).

To explore the consequences of the observed communication between macrophages and cardiomyocytes, mathematical modeling of electrical interactions between macrophages and AV cardiomyocytes was pursued (see Table 1 for model parameters). Recapitulating the experimental data (FIG. 4I), modeling indicates that the cardiomyocyte resting membrane potential is more depolarized when the cell is coupled to a macrophage, an effect that increases with gap junction conductance (FIG. 11F).

Modeling suggests that coupling increasing numbers of macrophages accelerates cardiomyocyte repolarization (FIG. 4J). For example, coupling three macrophages to an AV bundle cardiomyocyte, a ratio supported by histology (3±0.3, mean±SEM, n=17 in 5 mice; FIGS. 1 and 4C), decreases cardiomyocyte action potential duration from 30 ms to 21 ms while depolarizing the resting membrane potential from −69 mV to −52 mV (FIGS. 4K and 4L), assuming a gap junction conductance of 1 nS. In vivo, a shorter action potential duration would decrease the effective refractory period of the myocyte and increase the frequency at which it can be depolarized. A higher resting membrane potential would facilitate depolarization with less stimulation. Both alterations facilitate AV conduction at higher frequencies. These results correspond well with prior conceptual models of electrotonic interactions between cardiomyocytes and electrically non-excitable cells.

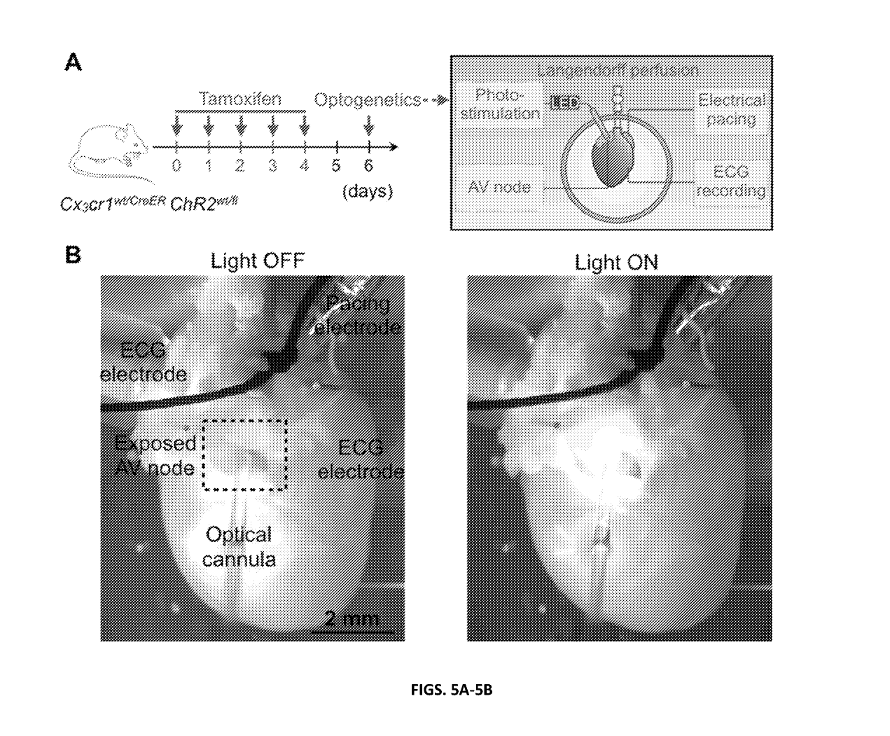

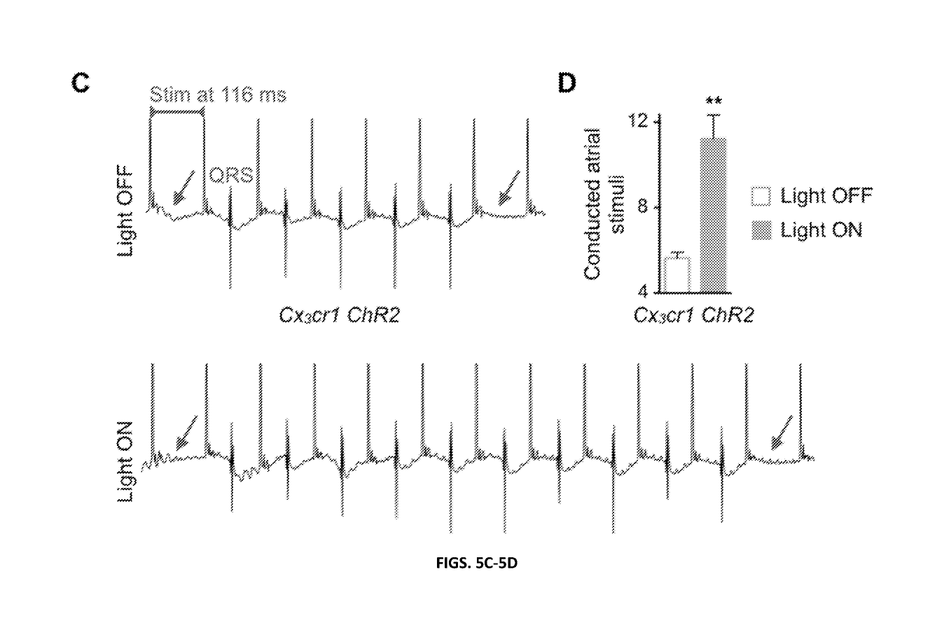

To investigate cell-cell communication directly in the AV node, photoactivatable channelrhodopsin 2 (ChR2) was expressed (43) in macrophages to control their membrane potential. When illuminated, the cation channel ChR2 undergoes a conformational change, resulting in an immediate increase in ionic permeability with high conductance for Na⁺ (44). The light-triggered cation influx was posited into macrophages and their resulting depolarization should alter AV node conduction if the cells are electrotonically coupled to conducting cardiomyocytes. To this end, tamoxifen-inducible Cx₃cr1^{CreER} were bred with ChR2V mice to obtain mice in which tamoxifen treatment triggers ChR2 expression in macrophages, hereafter denoted Cx₃cr1 ChR2. First, macrophage-specific expression of the tamoxifen-inducible Cre recombinase fusion protein (CreER) was validated by measuring YFP fluorescence in heart tissue, as YFP is co-expressed with CreER It was found that YFP signal colocalizes with CX₃CR1⁺ macrophages whereas cardiomyocytes are YFP negative (FIG. 12A). In addition, after tamoxifen treatment, AV node macrophages specifically expressed the ChR2 protein, which is fused with YFP. Then, hearts isolated from Cx₃cr1 ChR2 mice were retrogradely perfused and a fiber optic cannula was inserted into the right atrium to directly illuminate the AV node region (see FIGS. 5A and 5B for experimental setup). AV node conduction was assessed by ECG during rapid electrical atrial pacing, comparing continuous 470 nm wavelength illumination with no illumination. To evaluate the effect of ChR2-induced depolarization of macrophages on AV node function with high temporal resolution, the conducted atrial stimuli were counted between two non-conducted impulses during rapid pacing-induced Wenckebach block. Improved AV node conduction was observed during photostimulation of macrophages in hearts harvested from Cx₃cr1 ChR2 mice (n=5).

When the light was switched on, the number of conducted atrial stimuli between two non-conducted impulses rose (FIGS. 5C and 5D). In Cx₃cr1^{wt/CreER} control hearts (n=3), no difference was observed between illuminated and non-illuminated states. Thus, opening the cation channel ChR2 in macrophages facilitates AV node conduction during rapid pacing. Modeling indicates that with ChR2-induced tonic depolarization of macrophages, the minimum heterocellular coupling required to achieve macrophage-mediated passive action potential conduction between otherwise not connected cardiomyocytes becomes smaller (FIGS. 12B and 12C). Taken together, these observations suggest that cardiac macrophages can electrically couple to cardiomyocytes via gap junctions containing Cx43. This leads to cyclical macrophage depolarization, modulates cardiomyocytes' electrophysiological properties and alters AV nodal conduction.

### Example 4: Deleting Cx43 in Macrophages Delays AV Conduction

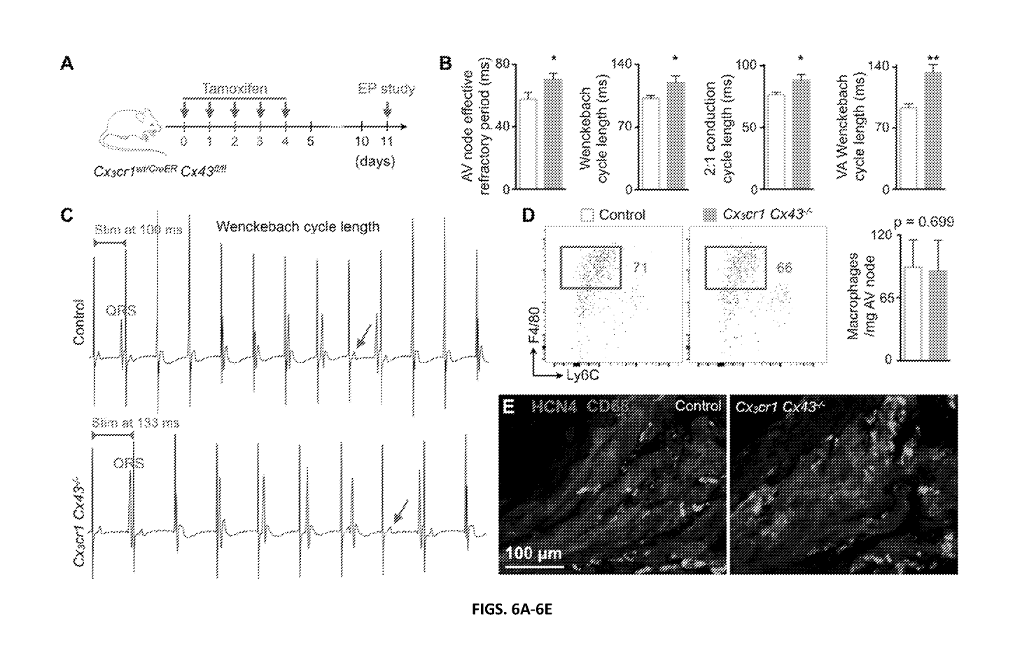

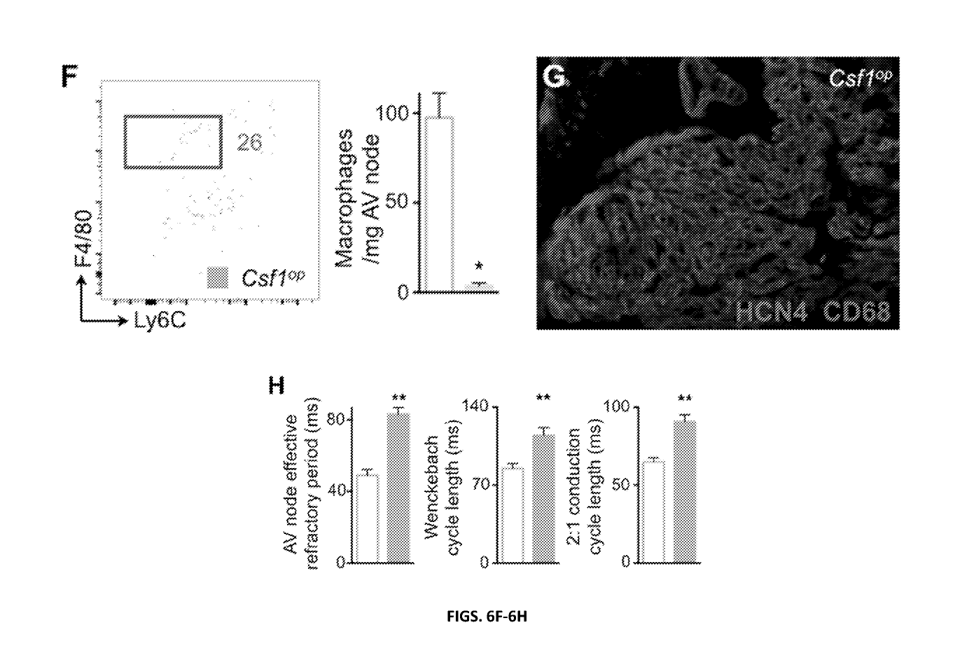

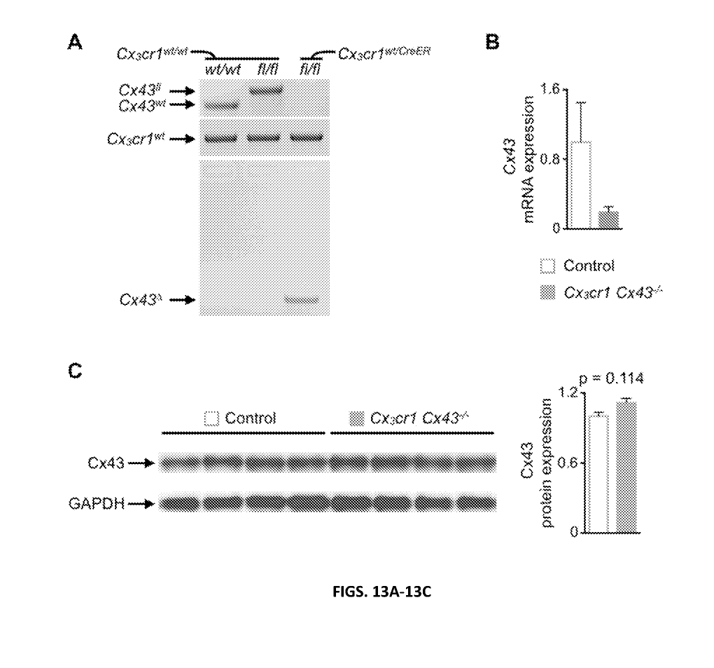

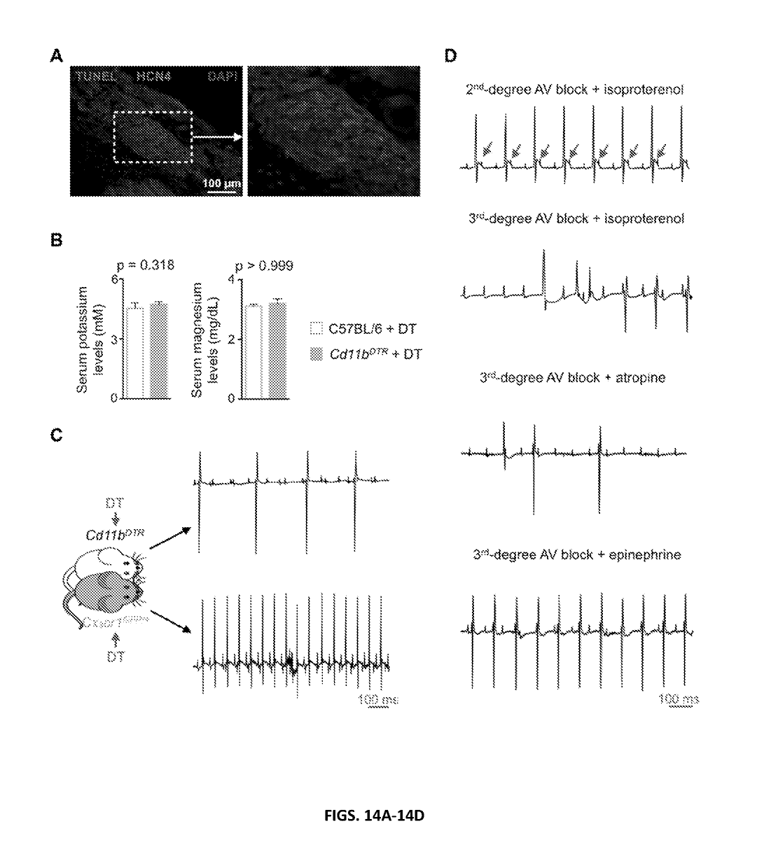

Examples 1-3 indicate that macrophages present in the AV node may facilitate conduction. To test this hypothesis in loss-of-function experiments, and to directly investigate the importance of Cx43 in macrophages, mice were bred in which tamoxifen treatment deleted Cx43 in CX₃CR1-expressing cells, hereafter denoted Cx₃cr1 Cx43f. In the AV node, all CX₃CR1⁺ cells are macrophages (FIGS. 2 and 10A). All mice underwent analysis seven days after tamoxifen treatment (FIG. 6A). Genomic PCR-based examination of the wild-type (Cx43^{wt}), floxed intact (Cx43^{fl}) and recombined (Cx43^{Δ}) alleles of the Cx43 gene in FACS-purified CX₃CR1⁺ cardiac macrophages showed effective Cx43 deletion in cardiac macrophages after tamoxifen treatment (FIG. 13A). mRNA analysis supported these findings (FIG. 13B). The overall myocardial Cx43 protein level did not change, indicating unaltered Cx43 expression in other cardiac cells (FIG. 13C).

To determine how macrophage-specific Cx43 deletion affects AV nodal function, an in vivo electrophysiological (EP) study was performed on Cx₃cr1 Cx43^{-/-} mice and littermate controls. The AV node effective refractory period was prolonged in Cx₃cr1 Cx43^{-/-} mice (FIG. 6B). Three additional parameters of AV nodal function were examined including the pacing cycle lengths at which Wenckebach conduction, 2:1 conduction and ventriculo-atrial Wenckebach conduction occur. In Cx₃cr1 Cx43^{-/-} mice, each of these parameters was prolonged, indicating impaired AV conduction (FIG. 6B). Representative surface ECG tracings of an AV Wenckebach block in control and Cx₃cr1 Cx43^{-/-} mice are shown in FIG. 6C. There is progressive PR prolongation prior to AV block, which develops at a slower pacing rate in Cx₃cr1 Cx43^{-/-} mice compared to controls. We did not observe differences in sinus node function or atrial refractory period (Table 2), and compromised AV conduction in Cx₃cr1 Cx43^{-/-} mice was not accompanied by altered AV node macrophage numbers (FIGS. 6D and 6E). These data indicate that macrophage Cx43 facilitates AV node conduction.

To explore the effect of congenital macrophage loss on AV node conduction, an EP study in Csf1^{op} mice, which lack Csf1-dependent tissue macrophages in many organs (45), was performed. The absence of AV node macrophages in Csf1^{op} mice (FIGS. 6F and 6G) prolonged the AV node effective refractory period as well as the pacing cycle lengths at which Wenckebach conduction and 2:1 conduction occurred (FIG. 6H). Interestingly, an increase in the atrial refractory period of Csf1^{op} mice was also observed (Table 2).

TABLE 2
Sinus Node Function and Atrial Characteristics of Control,
Cx₃cr1 Cx43^{-/-} and Csf1^{op} Mice by in vivo EP Study.

| | Control | Cx₃cr1 Cx43^{-/-} | | Control | Csf1^{op} | |
|---|---|---|---|---|---|---|
| | (n = 9) | (n = 8) | p value | (n = 6) | (n = 5) | p value |
| Sinus Node Function | | | | | | |
| SNRT/BCL_{120 ms} | 229.0 ± 16.1 | 207.8 ± 30.5 | 0.533 | 159.2 ± 15.3 | 176.8 ± 19.6 | 0.571 |
| SNRT/BCL_{100 ms} | 162.9 ± 8.6 | 184.5 ± 16.6 | 0.251 | 128.3 ± 15.4 | 163.8 ± 19.2 | 0.227 |
| SNRT/BCL_{80 ms} | 148.0 ± 15.0 | 162.6 ± 24.1 | 0.605 | 123.8 ± 18.1 | 161.4 ± 21.0 | 0.247 |
| Atrial Characteristics | | | | | | |
| AERP_{120 ms} | 34.6 ± 2.8 | 32.7 ± 2.2 | 0.611 | 36.0 ± 2.0 | 50.8 ± 3.6 | 0.016 |
| AERP_{100 ms} | 36.9 ± 2.9 | 38.9 ± 3.3 | 0.657 | 33.0 ± 3.0 | 49.0 ± 4.8 | 0.024 |

Data are mean ± SEM from 2 (Cx₃cr1 Cx43^{-/-}) and 3 (Csf1^{op}) independent experiments, Student's t test (Cx₃cr1 Cx43^{-/-}) and nonparametric Mann-Whitney test (Csf1^{op}).
AERP, atrial effective refractory period; BCL, basic cycle length; SNRT, sinus node recovery time.

### Example 5: Macrophage Ablation Induces AV Block

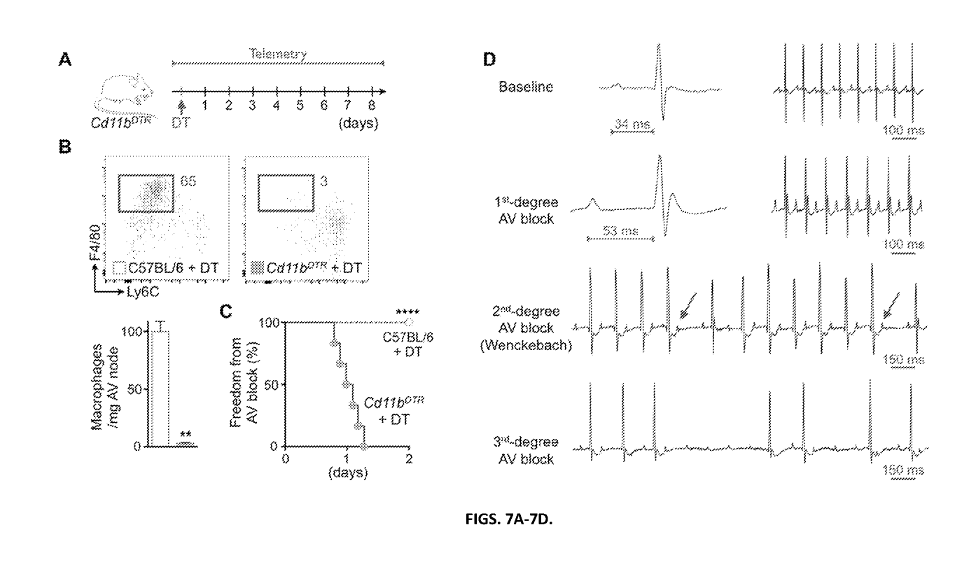

Cd11b^{DTR} mice express a diphtheria toxin (DT)-inducible system controlled by the human CD11b promoter that enables efficient depletion of myeloid cells, including resident cardiac macrophages (12). These mice were monitored continuously by implantable ECG telemetry after macrophage ablation (FIG. 7A). Maximum depletion of AV node macrophages happened three days after a single dose of 25 ng/g body weight DT (FIG. 7B). Within one day of DT injection, all mice developed first degree AV block (FIG. 7C) that progressively evolved into second and third degree AV block (FIG. 7D). Complete AV block coincided with the time point of peak AV node macrophage depletion. AV block after depletion of macrophages in Cd11b^{DTR} mice has not been previously reported, since ECG is not commonly monitored in immunological studies.

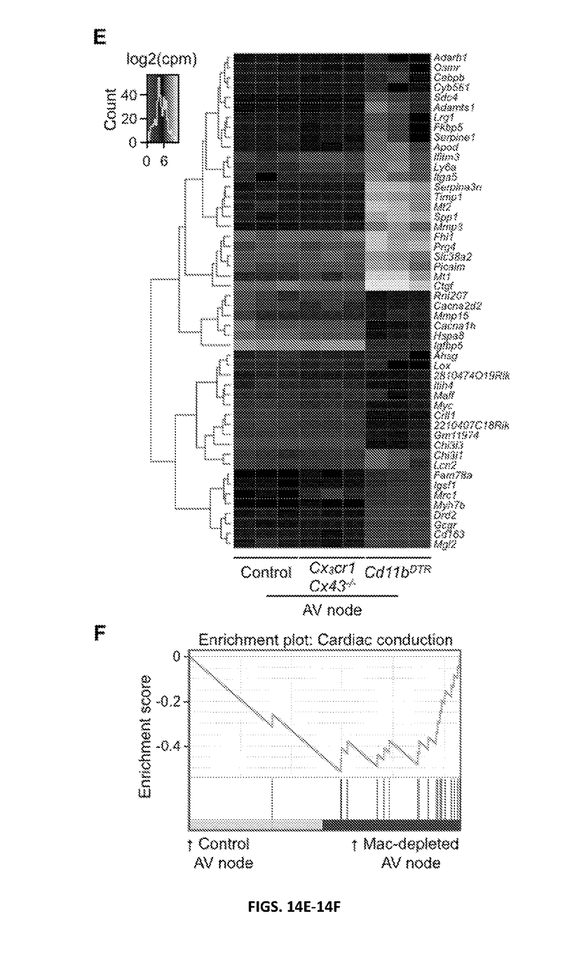

To determine whether the observed phenotype resulted from DT-related toxicity, C57BL/6 mice were injected with DT and their surface ECG was monitored. DT did not alter the number of AV node macrophages in C57BL/6 mice (FIG. 7B) and did not induce AV block (FIG. 7C). At the time of complete AV block, increased myocyte death in AV nodes of Cd11b^{DTR} mice was not observed (FIG. 14A). Because blood electrolyte levels may influence conduction, serum potassium and magnesium levels were measured, which were unchanged in mice with AV block (FIG. 14B). Moreover, DT did not induce AV block in Cx₃cr1^{GFP/+} mice joined in parabiosis with Cd11b^{DTR} mice, which developed AV block while in parabiosis, thereby indicating that circulating factors do not contribute to the observed phenotype (FIG. 14C). Injections of isoproterenol, epinephrine and atropine did not attenuate the AV block (FIG. 14D). This suggests that the AV block induced by macrophage ablation did not result from imbalanced autonomic nervous control.

When macrophages were depleted with clodronate liposomes (13), flow cytometry of microdissected AV nodes indicated incomplete macrophage depletion in this tissue (37% decrease in AV node macrophages). No AV node conduction abnormalities were observed by ECG telemetry and EP study. The absence of an AV node phenotype when using clodronate liposomes is likely due to the incomplete depletion of tissue-resident macrophages in the AV node.

Three loss-of-function experiments indicate that macrophages facilitate AV node conduction; however, the observed phenotypes differ in their severity. To better understand the observed differences, the whole transcriptome of AV node tissue microdissected from control, Cx₃cr1 Cx43^{-/-} and macrophage-depleted Cd11b^{DTR} hearts were compared by RNA-seq. The transcriptional profile of Cx₃cr1 Cx43^{-/-} AV nodes resembled control nodal tissue with only four genes significantly dysregulated while macrophage depletion led to a distinct expression profile characterized by 1,329 differentially expressed genes (FDR<0.05; FIG. 14E and Table 3). Genes associated with cardiac conduction are expressed at lower levels in macrophage-depleted AV nodes than in controls (FIG. 14F). Thus, deletion of Cx43 in macrophages had mild effects, while depletion of the cells changed the AV node expression profile, and consequently its function, more drastically. These data suggest that AV node macrophages engage in additional, Cx43 independent tasks, which may or may not be related to conduction.

TABLE 3
Differentially Expressed Genes in Cx₃cr1 Cx43^{-/-} AV Nodes
Compared with Control AV Nodes

| Gene | log2(FC) | log2 (cpm) | p value | FDR |
|---|---|---|---|---|
| Cytl1 | −1.31 | 8.12 | 1.15E−10 | 1.52E−06 |
| Vwf | 1.27 | 7.25 | 2.29E−08 | 1.52E−04 |
| Eln | 1.01 | 7.04 | 3.97E−06 | 1.05E−02 |
| Chi3l3 | 3.13 | 4.43 | 1.98E−05 | 4.38E−02 |

n = 3 per group. cmp, counts per million;
FC, fold change;
FDR, false discovery rate.

### REFERENCES AND NOTES

1. É. Metchnikoff, Leçons sur la pathologie comparée de l'inflammation (Masson, Paris, 1892).
2. Y. Lavin et al., Tissue-resident macrophage enhancer landscapes are shaped by the local microenvironment. Cell 159, 1312-1326 (2014).
3. K. D. Nguyen et al., Alternatively activated macrophages produce catecholamines to sustain adaptive thermogenesis. Nature 480, 104-108 (2011).
4. M. Kohyama et al., Role for spi-c in the development of red pulp macrophages and splenic iron homeostasis. Nature 457, 318-321 (2009).
5. R. C. Paolicelli et al., Synaptic pruning by microglia is necessary for normal brain development. Science 333, 1456-1458 (2011).
6. L. C. Davies, S. J. Jenkins, J. E. Allen, P. R Taylor, Tissue-resident macrophages. Nat Immunol 14, 986-995 (2013).
7. S. Tawara, Das reizleitungssystem des säugetierherzens: Eine anatomisch-histologische studie über das atrioventrikularbiindel und der purkinjeschen fäden (Verlag von Gustav Fischer, Jena, 1906).
8. A. de Carvalho, D. de Almeida, Spread of activity through the atrioventricular node. Circ Res 8, 801-809 (1960).
9. J. Billette, Atrioventricular nodal activation during periodic premature stimulation of the atrium. Am J Physiol 252, H163-77 (1987).
10. M. Rubart, D. P. Zipes, in Arrhythmias, sudden death and syncope, P. Libby, R. O. Bonow, D. L. Mann, D. Zipes, Eds. (Suanders Elsevier, Philadelphia, 2008), pp. 909-921.
11. A. R Pinto et al., An abundant tissue macrophage population in the adult murine heart with a distinct alternatively-activated macrophage profile. PLoS One 7, e36814 (2012).
12. T. Heidt et al., Differential contribution of monocytes to heart macrophages in steady-state and after myocardial infarction. Circ Res 115, 284-295 (2014).
13. S. Epelman et al., Embryonic and adult-derived resident cardiac macrophages are maintained through distinct mechanisms at steady state and during inflammation. Immunity 40, 91-104 (2014).
14. F. K. Swirski, M. Nahrendorf, Leukocyte behavior in atherosclerosis, myocardial infarction, and heart failure. Science 339, 161-166 (2013).
15. V. M. Unger, N. M. Kumar, N. B. Gilula, M. Yeager, Three-dimensional structure of a recombinant gap junction membrane channel. Science 283, 1176-1180 (1999).
16. M. Rosas et al., The transcription factor gata6 links tissue macrophage phenotype and proliferative renewal. Science 344, 645-648 (2014).
17. S. Rohr, Role of gap junctions in the propagation of the cardiac action potential. Cardiovasc Res 62, 309-322 (2004).
18. J. M. Nerbonne, C. G. Nichols, T. L. Schwarz, D. Escande, Genetic manipulation of cardiac k(+) channel function in mice: What have we learned, and where do we go from here. Circ Res 89, 944-956 (2001).
19. E. K. Gallin, J. I. Gallin, Interaction of chemotactic factors with human macrophages. Induction of transmembrane potential changes. J Cell Biol 75, 277-289 (1977).
20. E. K. Gallin, D. R Livengood, Nonlinear current-voltage relationships in cultured macrophages. J Cell Biol 85, 160-165 (1980).
21. F. V. McCann, J. J. Cole, P. M. Guyre, J. A. Russell, Action potentials in macrophages derived from human monocytes. Science 219, 991-993 (1983).
22. H. J. Woehlck, F. V. McCann, Action potentials in human macrophages are calcium spikes. Cell Biol Int Rep 10, 517-525 (1986).
23. J. Neijssen et al., Cross-presentation by intercellular peptide transfer through gap junctions. Nature 434, 83-88 (2005).
24. E. Mazzini, L. Massimiliano, G. Penna, M. Rescigno, Oral tolerance can be established via gap junction transfer of fed antigens from cx3cr1⁺ macrophages to cd103⁺ dendritic cells. Immunity 40, 248-261 (2014).
25. L. M. Davis, M. E. Rodefeld, K. Green, E. C. Beyer, J. E. Saffitz, Gap junction protein phenotypes of the human heart and conduction system. J Cardiovasc Electrophysiol 6, 813-822 (1995).
26. V. P. Nikolski, S. A. Jones, M. K. Lancaster, M. R Boyett, I. R Efimov, Cx43 and dual-pathway electrophysiology of the atrioventricular node and atrioventricular nodal reentry. Circ Res 92, 469-475 (2003).
27. W. J. Hucker, M. L. McCain, J. I. Laughner, P. A. Iaizzo, I. R Efimov, Connexin 43 expression delineates two discrete pathways in the human atrioventricular junction. Anat Rec (Hoboken) 291, 204-215 (2008).
28. S. A. Thomas et al., Disparate effects of deficient expression of connexin 43 on atrial and ventricular conduction: Evidence for chamber-specific molecular determinants of conduction. Circulation 97, 686-691 (1998).
29. C. Agulhon et al., What is the role of astrocyte calcium in neurophysiology. Neuron 59, 932-946 (2008).
30. O. Pascual, S. Ben Achour, P. Rostaing, A. Triller, A. Bessis, Microglia activation triggers astrocyte-mediated modulation of excitatory neurotransmission. Proc Natl Acad Sci USA 109, E197-205 (2012).
31. M. Zoob, K. S. Smith, The aetiology of complete heart-block. Br Med J 2, 1149-1153 (1963).
32. I. P. Moskowitz et al., A molecular pathway including id2, tbx5, and nkx2-5 required for cardiac conduction system development. Cell 129, 1365-1376 (2007).
33. R. A. Irizarry et al., Exploration, normalization, and summaries of high density oligonucleotide array probe level data. Biostatistics 4, 249-264 (2003).
34. G. Bu, H. Adams, E. J. Berbari, M. Rubart, Uniform action potential repolarization within the sarcolemma of in situ ventricular cardiomyocytes. Biophys J 96, 2532-2546 (2009).
35. A. Dobin et al., Bioinformatics 29, 15 (2013).
36. S. Anders, P. T. Pyl, W. Huber, Htseq-a python framework to work with high-throughput sequencing data. Bioinformatics 31, 166-169 (2015).
37. M. D. Robinson, D. J. McCarthy, G. K. Smyth, Edger: A bioconductor package for differential expression analysis of digital gene expression data. Bioinformatics 26, 139-140 (2010).
38. V. K. Mootha et al., Pgc-1alpha-responsive genes involved in oxidative phosphorylation are coordinately downregulated in human diabetes. Nat Genet 34, 267-273 (2003).
39. P. V. Kharchenko, L. Silberstein, D. T. Scadden, Bayesian approach to single-cell differential expression analysis. Nat Methods 11, 740-742 (2014).
40. F. B. Sachse, A. P. Moreno, J. A. Abildskov, Electrophysiological modeling of fibroblasts and their interaction with myocytes. Ann Biomed Eng 36, 41-56 (2008).
41. S. Inada, J. C. Hancox, H. Zhang, M. R Boyett, One-dimensional mathematical model of the atrioventricular node including atrio-nodal, nodal, and nodal-his cells. Biophys J 97, 2117-2127 (2009).
42. H. Xu, H. Li, J. M. Nerbonne, Elimination of the transient outward current and action potential prolongation in mouse atrial myocytes expressing a dominant negative kv4 alpha subunit. J Physiol 519 Pt 1, 11-21 (1999).
43. L. Fenno, O. Yizhar, K. Deisseroth, The development and application of optogenetics. Annu Rev Neurosci 34, 389-412 (2011).
44. G. Nagel et al., Channelrhodopsin-2, a directly light-gated cation-selective membrane channel. Proc Natl Acad Sci USA 100, 13940-13945 (2003).
45. M. G. Cecchini et al., Role of colony stimulating factor-1 in the establishment and regulation of tissue macrophages during postnatal development of the mouse. Development 120, 1357-1372 (1994).

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A composition comprising a macrophage-targeted carrier and one or more therapeutic agents that modulate cardiac conductance, and optionally a pharmaceutically acceptable carrier.

2. The composition of claim 1, wherein the macrophage-targeted carrier is selected from the group consisting of microspheres/microparticles, liposomes, lipid nanoparticles, carbohydrate nanoparticles, dendrimers, exosomes, extracellular vesicles, carbon nanotubes, and polymersomes.

3. The composition of claim 1, wherein the therapeutic agent decreases conductance.

4. The composition of claim 3, wherein the therapeutic agent decreases gap junction communication.

5. The composition of claim 4, wherein the agent is endothelin-1, angiotensin II, Rotigaptide (ZP-123), peptide VCYDKSFPISHVR (SEQ ID NO: 1) corresponding to AA63-75 of E1 of Cx43; peptide SRPTEKTIFII (SEQ ID NO:2) corresponding to AA204-214 of E2 of Cx43; peptide KRDPCHQVDCFLSRPTEK (SEQ ID NO:3) corresponding to AA191-209 of E2 of Cx43), peptide AAP10 (H-Gly-Ala-Gly-Hyp-Pro-Tyr-CONH2), SEQ ID NO:4, cAAP10RG, AAPnat, and gap-134.

6. The composition of claim 3, wherein the therapeutic agent is an anti-arrhythmic drug.

7. The composition of claim 6, wherein the anti-arrhythmic drug is a Ca²⁺ channel blocker; Na⁺ channel blocker; beta-adrenoceptor antagonists (beta-blockers); potassium-channel blocker; digoxin; or digitalis.

8. The composition of claim 1, wherein the therapeutic agent increases conductance.

9. The composition of claim 8, wherein the therapeutic agent is epinephrine, norepinephrine, dopamine, denopamine, dobutamine, salbutamol, atropine, isoproterenol, NS11021, naltriben, midefradil and NNC 50-0396, ICA-105574, PD-118057, NS1643, Pinacidil, 2-anilino-5-(2,4-dinitroanilino)benzenesulfonate; potassium channel agonists, optionally NS-1619,1-EBIO, minoxidil, cromakalim, or levcromakalim, or a cation, optionally K⁺, Na⁺, Ca²⁺, or Mg².

10. A method for treating a subject having a cardiac rhythm disorder, the method comprising administering to the subject a therapeutically effective amount of the composition of claim 1.

11. A method for treating a subject having tachycardia, comprising administering the composition of claim 4.

12. A method for treating a subject having tachycardia, comprising administering the composition of claim 5.

13. A method for treating a subject having bradycardia or a conductance block, comprising administering the composition of claim 8.

14. A method for treating a subject having bradycardia or a conductance block, comprising administering the composition of claim 9.

15.-19. (canceled)
